(19) United States
(12) Patent Application Publication
(10) Pub. No.: US 2011/0136877 A1
(43) Pub. Date: Jun. 9, 2011

(54) 2-PHENYL PHENOXYACETIC ACIDS USEFUL FOR TREATING INFLAMMATORY DISORDERS

(75) Inventors: Brian F. McGuinness, Plainsboro, NJ (US); Thuy X. H. Le, Monmouth Junction, NJ (US); Jingqi Duo, Paoli, PA (US); Koc-Kan Ho, Salt Lake City, UT (US)

(73) Assignee: LIGAND PHARMACEUTICALS INC., San Diego, CA (US)

(21) Appl. No.: 12/811,823

(22) PCT Filed: Jan. 6, 2009
(86) PCT No.: PCT/US2009/030172

§ 371 Date: Nov. 19, 2010

Related U.S. Application Data
(60) Provisional application No. 61/019,361, filed on Jan. 7, 2008.

(51) Int. Cl. A61K 31/4406; C07C 271/22; A61K 31/27; C07D 213/71; C07C 311/16; A61K 31/18; C07D 213/56; A61K 31/455; C07D 213/55; C07C 229/34; A61K 31/195; C07D 277/28; A61K 31/426; A61P 11/00; A61P 17/06; A61P 27/02; A61P 17/02; A61P 19/02; A61P 1/00; A61P 29/00

(52) U.S. Cl. 514/346; 560/27; 514/486; 546/293; 514/351; 564/92; 514/604; 546/316; 514/355; 546/291; 562/448; 514/564; 548/204; 514/365

(57) ABSTRACT

The present invention provides novel phenoxyacetic acids useful for the prevention and treatment of inflammatory disorders, including those affecting the respiratory system and skin. The compounds are of the general formula (I):

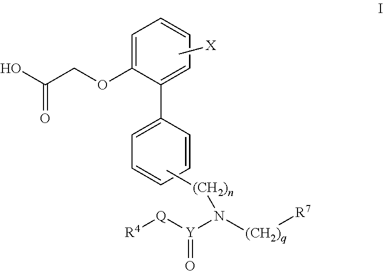

### FIELD OF THE INVENTION

The invention relates to 2-phenyl phenoxyacetic acids useful for treating inflammatory disorders.

### BACKGROUND OF THE INVENTION

CRTH2 (Chemoattractant Receptor-homogolous molecule expressed on T Helper 2 cells, also known as DP2) is a G protein coupled receptor expressed on the major pro-inflammatory cells: eosinophils, T-Helper 2 (TH2), and basophils. Its endogenous ligand Prostaglandin D₂ (PGD₂) is derived from arachidonic acid by sequential actions of cyclooxygenase and PGD₂ synthases. It has been reported that CRTH2, upon activation by PGD₂, leads to a number of inflammatory responses, which includes eosinophil shape change and degranulation (Gervais et al., 2001, J. Allergy Clin. Immunol. 108, 982-988), basophil degranulation (Yoshimura-Uchiyama et al., 2004, Clin. Exp. Allergy 34, 1283-1290), TH2 cell cytokine secretion (Tanaka et al., 2004, Biochem. Biophys. Res. Commun. 316, 1009-1014) and TH2 cell chemotaxis (Gyles et al., 2006, Immunology 119, 362-368). CRTH2 genetic knock-out (KO) data has been reported. CRTH2 KO mice show a significant decrease in antigen-induced lung inflammation (Chevalier et al., 2005, J. Immunolo. 175, 2056-2060). In addition to the KO data, Ramatroban, a marketed drug in Japan, has established efficacy against allergic rhinitis and is currently in clinical trial for treatment of asthma. Although the compound was first developed as a thromboxan antagonist, recent studies show that Ramatroban is also a potent CRTH2 antagonist (Pettipher et al., 2007, Nature Reviews Drug Discovery 6, 313-325). It has been suggested that the efficacy of Ramatroban in asthmatic and allergic reactions is in part mediated through CRTH2. A compound closely related to Ramatroban, TM30089, has been shown to reduce the pathology of asthma in vivo (Uller et al., 2007, Respiratory Research 8: 16).

Blockage of CRTH2, therefore, presents an attractive approach to treat various PGD₂-mediated inflammatory diseases. Among disorders in which PGD₂ is implicated are respiratory disorders, skin disorders, and other disorders related to allergic reactions.

CRTH2 is also expressed in the central nervous system (Nagata et al., 2003 Prostaglandins, Leukotrienes and Essential Fatty Acids 69, 169-177). CRTH2 mRNA was detected in various brain regions including the thalamus, frontal cortex, pons, hippocampus, hypothalamus, and caudate/putamen (Marchese et al., 1999 Genomics 56, 12-21). Corradini et al. (WO2005/102338) disclosed that small molecule antagonists of the CRTH2 receptor are efficacious in two rat models: the chronic constrictive injury model and Seltzer model. The data established a link between CRTH2 and pain.

Blockage of CRTH2, therefore, presents an attractive approach to treat various pain conditions such as neuropathic pain.

### SUMMARY OF THE INVENTION

It has now been found that compounds of general formula I are potent inhibitors of chemoattractant receptor-homogolous molecule expressed on T helper 2 cells (CRTH2):

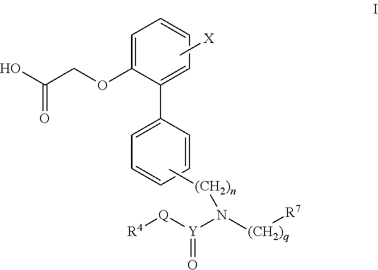

In these compounds, or a salt thereof,
X is selected from the group consisting of hydrogen, halogen, cyano, (C₁-C₄)alkyl, —O(C₁-C₄)alkyl and —S(O)ₘ(C₁-C₄)alkyl, each (C₁-C₄)alkyl optionally substituted with one chlorine, iodine or bromine atom or one or more fluorine atoms;
m is zero, one or two;
n is one or two;
Y is carbon or SO;
Q is oxygen, NH or (CH₂)ₚ, with the proviso that when Y is SO, Q cannot be oxygen;
p is zero or 1-4;
R⁴ is selected from the group consisting of:
   (a) aryl and heterocyclyl, each optionally substituted with one to three substituents from the group consisting of (C₁-C₄)alkyl, (C₁-C₄)alkoxy, (C₁-C₄)haloalkyl, halogen, cyano and (C₁-C₄)haloalkoxy;
   (b) (C₁-C₈)alkyl, optionally substituted with one to three substituents chosen from the group consisting of (C₁-C₈)alkyl, (C₁-C₈)haloalkyl, chlorine, iodine, bromine, cyano and (C₁-C₈)haloalkoxy or optionally substituted with one or more fluorine atoms; and
   (c) (C₁-C₈)heteroalkyl;
q is zero or 1-4; and
R⁷ is chosen from:
   (a) aryl and heterocyclyl, each optionally substituted with one to three substituents from the group consisting of (C₁-C₄)alkyl, (C₁-C₄)alkoxy, (C₁-C₄)haloalkyl, halogen, cyano and (C₁-C₄)haloalkoxy;
   (b) (C₁-C₈)alkyl, optionally substituted with one to three substituents from the group consisting of (C₁-C₈)alkyl, (C₁-C₈)haloalkyl, chlorine, iodine, bromine, cyano and (C₁-C₈)haloalkoxy or optionally substituted with one or more fluorine atoms;
   (c) (C₁-C₈)heteroalkyl; and
   (d) CON(H)(C₁-C₈)alkyl.

The members of this genus are useful in inhibiting CRTH2 activity and as such are potentially useful in indications where the suppression of the inflammatory response is desired.

In still another aspect, the invention relates to a pharmaceutically acceptable salt of a compound of the invention.

In another aspect, the invention relates to pharmaceutical compositions comprising a therapeutically effective amount of at least one compound of general formula I, or a pharmaceutically acceptable salt thereof, and a pharmaceutically acceptable carrier.

In another aspect, the invention relates to a method for treating, preventing or ameliorating a disorder by altering a response mediated by CRTH2. The method comprises bringing into contact with CRTH2 at least one compound or salt of general formula I.

In yet another aspect the present invention relates to a method of suppressing the inflammatory response in a subject in need thereof comprising administering to the subject a therapeutically effective amount of at least one compound or salt of general formula I. In certain embodiments, the subject is a human.

Suppression of the inflammatory response is desirable for controlling the body's extreme reaction to internal or external stimuli, such as that found with respiratory disorders, skin disorders and those disorders with an allergic component. Exemplary disorders include asthma, rhinitis, chronic obstructive pulmonary disorder, cough, bronchitis, dermatitis, psoriasis, pruritis and urticaria.

Other indications in which the CRTH2 inhibitors are useful include osteoarthritis, rheumatoid arthritis, conjunctivitis, corneal ulcers, chronic skin ulcers, inflammatory bowel disease and pain.

In yet another aspect, the invention relates to the use of a compound or salt of the invention for the treatment, prevention, or amelioration of a disorder responsive to inhibition of chemoattractant receptor-homogolous molecule expressed on T helper 2 cells. Examples of these disorders may be inflammatory disease or respiratory disease, such as asthma, rhinitis, chronic obstructive pulmonary disease, bronchitis, nasal polyposis, nasal congestion, farmer's lung, fibroid lung and cough. Further examples include skin disorders, such as dermatitis, cutaneous eosinophilias, Lichen planus, urticaria, psoriasis, pruritus, angiodermas, chronic skin ulcers, vasculitides or erythemas. Other examples of these disorders include corneal ulcers, conjunctivitis, uveitis, osteoarthritis, rheumatoid arthritis, pain and inflammatory bowel disease.

In still another aspect, the invention relates to the use of a compound or salt of the invention in the manufacture of a medicament for the treatment, prevention or amelioration of a disorder responsive to inhibition of chemoattractant receptor-homogolous molecule expressed on T helper 2 cells in a subject in need thereof. Examples of these disorders may be inflammatory disease or respiratory disease, such as asthma, rhinitis, chronic obstructive pulmonary disease, bronchitis, nasal polyposis, nasal congestion, farmer's lung, fibroid lung and cough. Further examples include skin disorders, such as dermatitis, cutaneous eosinophilias, Lichen planus, urticaria, psoriasis, pruritus, angiodermas, chronic skin ulcers, vasculitides or erythemas. Other examples of these disorders include corneal ulcers, conjunctivitis, uveitis, osteoarthritis, rheumatoid arthritis, pain and inflammatory bowel disease.

### DETAILED DESCRIPTION OF THE INVENTION

Throughout this specification the substituents are defined when introduced and retain their definitions.

In a first aspect the invention relates to 2-phenyl phenoxyacetic acids having general formula I:

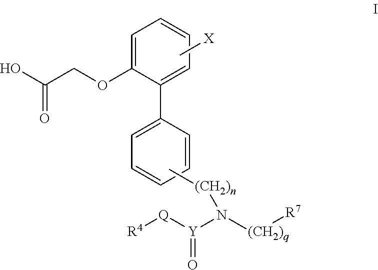

The members of the genus I may be conveniently divided into two subgenera based on the values of Y. When Y is equal to carbon, a subgenus of 2-phenyl phenoxyacetic acids having an attached carboxamide, urea or carbamate arises. When Y is SO, a subgenus of 2-phenyl phenoxyacetic acids having an attached sulfonamide or sulfonylurea arises. The structures of these subgenera are shown below:

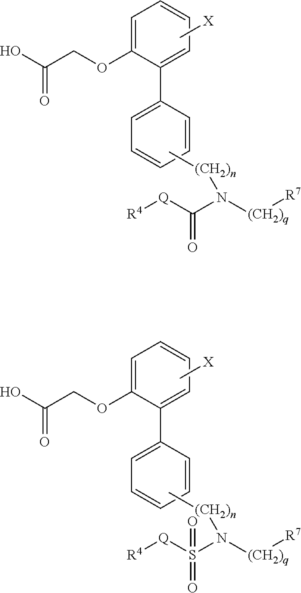

In certain embodiments, n may be equal to one. In other embodiments, n may be equal to two. In further embodiments, the CH₂ substituent can be in the meta position. In still other embodiments, the CH₂ substituent may be in the para position. In yet other embodiments, n may be equal to one and the CH₂ substituent can be in the meta position. In further embodiments, n may be equal to two and the CH₂ substituent can be in the para position.

In certain embodiments, Q is oxygen. In other embodiments, Q is equal to NH. In further embodiments, Q can be (CH₂)ₚ and p can be zero, one, two, three or four. In some embodiments, R⁴ is a (C₁-C₈)alkyl, optionally substituted with one to three substituents chosen from the group consisting of (C₁-C₈)alkyl, (C₁-C₈)haloalkyl, chlorine, iodine, bromine, cyano and (C₁-C₈)haloalkoxy or optionally substituted with one or more fluorine atoms. In some other embodiments, R⁴ is an aryl or heterocyclyl, each optionally substituted with one to three substituents from the group consisting of (C₁-C₄)alkyl, (C₁-C₄)alkoxy, (C₁-C₄)haloalkyl, halogen, cyano and (C₁-C₄)haloalkoxy. For example, R⁴ may be a pyridinyl or phenyl group. In yet other embodiments, R⁴ is a (C₁-C₈)heteroalkyl.

In some embodiments, q is one. In other embodiments, q is zero. In yet other embodiments, R⁷ is CON(H)(C₁-C₈)alkyl. In still other embodiments R⁷ is aryl or heterocyclyl, each optionally substituted with one to three substituents chosen from (C₁-C₄)alkyl, (C₁-C₄)alkoxy, (C₁-C₄)haloalkyl, halogen, cyano and (C₁-C₄)haloalkoxy. For instance, R⁷ may be a thiazolyl, pyridinyl, or phenyl group. In further embodiments R⁷ is (C₁-C₈)alkyl, optionally substituted with one to three substituents chosen from the group consisting of (C₁-C₈)alkyl, (C₁-C₈)haloalkyl, chlorine, iodine, bromine, cyano and (C₁-C₈)haloalkoxy or optionally substituted with one or more fluorine atoms. In yet other embodiments, R⁷ is a (C₁-C₈)heteroalkyl.

In some embodiments, X may be hydrogen, halogen, cyano, (C₁-C₄)alkyl, —O(C₁-C₄)alkyl and —S(O)ₘ(C₁-C₄)alkyl, each (C₁-C₄)alkyl optionally substituted with one of chlorine, iodine or bromine, or one or more of fluorine, where m is zero, one or two. For example, X may be H, F, Cl, CH₃ or CF₃. In some embodiments, X is located at the 4-position of the phenyl labeled “a”.

In some embodiments, X is located at the 4-position relative to the oxyacetic acid substituent of the phenyl group labeled “a”, n is one, and the CH₂ substituent is meta on the phenyl group labeled “b”, resulting in compounds of general formula II

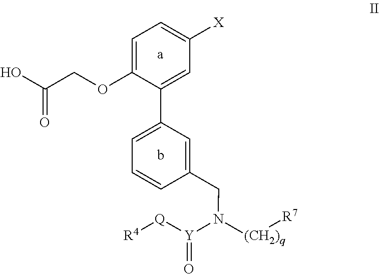

In yet other embodiments, Y is carbon, Q is oxygen, and R⁴ is a (C₁-C₈)alkyl, aryl or heteroaryl. In further embodiments, Y is carbon, Q is (CH₂)ₚ, p is zero or 1-4, and R⁴ is a (C₁-C₈)alkyl, aryl or heteroaryl. In yet other embodiments, Y is carbon, Q is NH, and R⁴ is a (C₁-C₈)alkyl, aryl or heteroaryl. In still further embodiments, Y is SO, Q is (CH₂)ₚ, p is zero or 1-4, and R⁴ is a (C₁-C₈)alkyl, aryl or heteroaryl.

In certain embodiments, q is one and R⁷ is CON(H)(C₁-C₈)alkyl. In other embodiments, q is one and R⁷ is (C₁-C₈)alkyl, aryl or heteroaryl. In still other embodiments, q is zero and R⁷ is aryl or (C₁-C₈)alkyl.

All of the compounds falling within the foregoing parent genus and its subgenera are useful as CRTH2 modulators. It may be found upon examination that compounds that have been excluded from the claims to compounds or compounds that have been excluded from the claims to methods are patentable to the inventors in this application; it may also be found that additional species and genera not presently excluded are not patentable to the inventors in this application. In either case, the exclusion of species and genera in applicants' claims are to be considered artifacts of patent prosecution and not reflective of the inventors' concept or description of their invention. The invention, in a composition aspect, is all compounds of formula I except those that are in the public's possession.

### DEFINITIONS

For convenience and clarity certain terms employed in the specification, examples and claims are described herein.

Alkyl is intended to include linear, branched, or cyclic hydrocarbon structures and combinations thereof. A combination would be, for example, cyclopropylmethyl. Lower alkyl refers to alkyl groups of from 1 to 6 carbon atoms. Examples of lower alkyl groups include methyl, ethyl, propyl (both n-propyl and isopropyl), butyl (including s- and t-butyl) and the like. Preferred alkyl and alkylene groups are those of C₂₀ or below; more preferred are C₁-C₈ alkyl; most preferred are C₁-C₄ alkyl. Cycloalkyl is a subset of alkyl and includes cyclic hydrocarbon groups of from 3 to 8 carbon atoms. Examples of cycloalkyl groups include cyclopropyl, cyclobutyl, cyclopentyl, cyclohexyl, norbornyl and the like.

C₁ to C₂₀ Hydrocarbon (e.g. C₁, C₂, C₃, C₄, C₅, C₆, C₇, C₈, C₉, C₁₀, etc.) includes alkyl, cycloalkyl, alkenyl, alkynyl, aryl and combinations thereof. Examples include benzyl, phenethyl, cyclohexylmethyl, camphoryl and naphthylethyl. Hydrocarbon refers to any substituent comprised of hydrogen and carbon as the only elemental constituents.

Alkoxy or alkoxyl refers to groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy.

Heteroalkyl refers to carbon-attached branched or linear alkyl residues in which one or more non-adjacent, non-terminal carbons (and their associated hydrogens) have been replaced by a heteroatom. For example, oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons have been replaced by sulfur or nitrogen, respectively. Examples include ethylaminoethyl and methylthiopropyl.

Acyl refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself, is called “oxo”.

Aryl and heteroaryl mean a 5- or 6-membered aromatic or heteroaromatic ring containing 0-4 heteroatoms selected from O, N, or S; a bicyclic 9- or 10-membered aromatic or heteroaromatic ring system containing 0-4 heteroatoms selected from O, N, or S; or a tricyclic 13- or 14-membered aromatic or heteroaromatic ring system containing 0-5 heteroatoms selected from O, N, or S. The aromatic 6- to 14-membered carbocyclic rings include, e.g., benzene, naphthalene, tetrahydronaphthalene (tetralin), indane and fluorene. The 5- to 10-membered aromatic heterocyclic rings include, e.g., imidazole, pyridine, indole, indoline, thiophene, benzopyranone, thiazole, furan, benzimidazole, benzodioxole, quinoline, isoquinoline, quinoxaline, pyrimidine, pyrazine, tetrazole and pyrazole. As used herein aryl refers to residues in which one or more rings are aromatic, but not all need be.

Arylalkyl refers to a substituent in which an aryl residue is attached to the parent structure through alkyl. Examples are benzyl, phenethyl and the like. This is in contradistinction to alkylaryl, in which an aryl residue is attached to the parent structure through alkyl (e.g. a p-tolyl residue). Heteroarylalkyl refers to a substituent in which a heteroaryl residue is attached to the parent structure through alkyl. Examples include, e.g., pyridinylmethyl, pyrimidinylethyl and the like. In one embodiment, the alkyl group of an arylalkyl or a heteroarylalkyl is an alkyl group of from 1 to 6 carbons.

The term “heterocycle” means a monocyclic, bicyclic or tricyclic residue with 1 to 13 carbon atoms and 1 to 4 heteroatoms chosen from the group consisting of nitrogen, oxygen and sulfur. The nitrogen and sulfur heteroatoms may optionally be oxidized, and the nitrogen heteroatom may optionally be quaternized. Unless otherwise specified, a heterocycle may be non-aromatic or aromatic. It is to be noted that heteroaryl is a subset of heterocycle in which the heterocycle is aromatic. The heterocycle may be fused to an aromatic hydrocarbon radical. Suitable examples include pyrrolyl, pyridinyl, pyrazolyl, triazolyl, pyrimidinyl, pyridazinyl, oxazolyl, thiazolyl, imidazolyl, indolyl, thiophenyl, furanyl, tetrazolyl, 2-pyrrolinyl, 3-pyrrolinyl, pyrrolindinyl, 1,3-dioxolanyl, imidazolinyl, imidazolidinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dithianyl, thiomorpholinyl, pyrazinyl, piperazinyl, 1,3,5-triazinyl, 1,2,5-trithianyl, benzo[b]thiophenyl, benzimidazolyl, quinolinyl, and the like. A nitrogen heterocycle is a heterocycle containing at least one nitrogen in the ring; it may contain additional nitrogens, as well as other heteroatoms. Examples include piperidine, piperazine, morpholine, pyrrolidine and thiomorpholine. It is to be noted that heteroaryl is a subset of heterocycle in which the heterocycle is aromatic; examples include pyridine, pyrrole and thiazole. Examples of heterocyclyl residues additionally include piperazinyl, 2-oxopiperazinyl, 2-oxopiperidinyl, 2-oxopyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

The term “carbocycle” is intended to include ring systems, including polycyclic structures, consisting entirely of carbon but of any oxidation state. Thus (C₃-C₁₀) carbocycle refers to such systems as cyclopropane, benzene and cyclohexene; (C₈-C₁₂) carbopolycycle refers to such systems as norbornane, decalin, indane and naphthalene. Carbocycle, not otherwise limited, refers to monocycles, bicycles and polycycles.

The terms “monocycle” and “bicycle” or “monocyclic” and “bicyclic” refer to carbocycles and heterocycles having one or two rings respectively. Preferred monocycles are 3, 4, 5, 6 or 7-membered rings, which may be aromatic, saturated or partially unsaturated. Non-limiting examples include cyclopropane, cyclopentane, cyclohexane, pyran, furan, tetrahydrofuran, tetrahydropyran, oxepane and phenyl. Preferred bicycles are those having from 8 to 12 ring atoms in total. Non-limiting examples include chroman, tetralin, naphthalene, benzofuran, indole, octahydropentalene and tetrahydrobenzo[b]oxepine. A particular embodiment comprises fused 5:6 and 6:6 systems.

As used herein, the term “optionally substituted” may be used interchangeably with “unsubstituted or substituted”. The term “substituted” refers to the replacement of one or more hydrogen atoms in a specified group with a specified radical. In one embodiment, 1, 2 or 3 hydrogen atoms are replaced with a specified radical. In the case of alkyl and cycloalkyl, more than three hydrogen atoms can be replaced by fluorine; indeed, all available hydrogen atoms could be replaced by fluorine.

The terms “halogen” and “halo” refer to fluorine, chlorine, bromine or iodine.

The terms “haloalkyl” and “haloalkoxy” mean alkyl or alkoxy, respectively, substituted with one or more halogen atoms. The terms “alkylcarbonyl” and “alkoxycarbonyl” mean —C(═O)alkyl or —C(O)alkoxy, respectively.

Substituents Rⁿ are generally defined when introduced and retain that definition throughout the specification and in all independent claims.

Some of the compounds described herein may contain one or more asymmetric centers and may thus give rise to enantiomers, diastereomers, and other stereoisomeric forms that may be defined, in terms of absolute stereochemistry, as (R)- or (S)-. The present invention is meant to include all such possible isomers, as well as mixtures thereof, including racemic and optically pure forms. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration; thus a carbon-carbon double bond depicted arbitrarily herein as trans may be Z, E or a mixture of the two in any proportion.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

It will be recognized that the compounds of this invention can exist in radiolabeled form, i.e., the compounds may contain an unnatural ratio of one or more atoms containing an atomic mass or mass number different from the atomic mass or mass number usually found in nature. Radioisotopes of hydrogen, carbon, phosphorous, fluorine, chlorine and iodine include ³H, ¹⁴C, ³⁵S, ¹⁸F, ³⁶Cl and ¹²⁵I, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention can generally be prepared by methods well known to those skilled in the art. Conveniently, such radiolabeled compounds can be prepared by carrying out the procedures disclosed in the Examples by substituting a readily available radiolabeled reagent for a non-radiolabeled reagent. Because of the high affinity for the CRTH2 enzyme active site, radiolabeled compounds of the invention are useful for CRTH2 assays.

### Chemical Synthesis

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by T. W. Greene [John Wiley & Sons, New York, 1991], which is incorporated herein by reference.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, for example in the case of suitably substituted phenoxyacetic esters, are either commercially available, synthesized as described in the examples or may be obtained by the methods well known to persons of skill in the art.

The present invention further provides pharmaceutical compositions comprising as active agents, the compounds described herein.

As used herein a “pharmaceutical composition” refers to a preparation of one or more of the compounds described herein, or physiologically acceptable salts or solvates thereof, with other chemical components such as physiologically suitable carriers and excipients.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Compounds that modulate the function of CRTH2 can be formulated as pharmaceutical compositions and administered to a mammalian subject, such as a human patient in a variety of forms adapted to the chosen route of administration, i.e., orally or parenterally, by intravenous, intramuscular, topical, transdermal or subcutaneous routes.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar or alginic acid or a salt thereof such as sodium alginate.

In addition, enteric coating may be useful as it is may be desirable to prevent exposure of the compounds of the invention to the gastric environment.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers.

In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds of the invention may be formulated in aqueous solutions, preferably in physiologically compatible buffers such as Hank's or Ringer's solution or physiological saline buffer. For transmucosal and transdermal administration, penetrants appropriate to the barrier to be permeated may be used in the composition. Such penetrants, including for example DMSO or polyethylene glycol, are known in the art.

For administration by inhalation, the compounds for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds, to allow for the preparation of highly concentrated solutions.

The compounds of the present invention may also be formulated in rectal compositions such as suppositories or retention enemas, using, e.g., conventional suppository bases such as cocoa butter or other glycerides.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on many factors including the subject being treated, the severity of the affliction, the manner of administration, the judgment of the prescribing physician. The compounds of the invention may be administered orally or via injection at a dose from 0.001 to 2500 mg/kg per day. The dose range for adult humans is generally from 0.005 mg to 10 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

As used herein, and as would be understood by the person of skill in the art, the recitation of “a compound” is intended to include salts, solvates and inclusion complexes of that compound. The term “solvate” refers to a compound of Formula I in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Inclusion complexes are described in Remington: The Science and Practice of Pharmacy 19th Ed. (1995) volume 1, page 176-177, which is incorporated herein by reference. The most commonly employed inclusion complexes are those with cyclodextrins, and all cyclodextrin complexes, natural and synthetic, are specifically encompassed within the claims.

The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, carbonic, citric, ethanedisulfonic, ethanesulfonic, ethylenediaminetetraacetic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, pamoic, pantothenic, phosphoric, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine.

The term “preventing” as used herein refers to administering a medicament beforehand to forestall or obtund an attack. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended herein.

It should be understood that in addition to the ingredients particularly mentioned above, the formulations of this invention may include other agents conventional in the art having regard to the type of formulation in question, for example those suitable for oral administration may include flavoring agents.

The compositions may be presented in a packaging device or dispenser, which may contain one or more unit dosage forms containing the active ingredient. Examples of a packaging device include metal or plastic foil, such as a blister pack and a nebulizer for inhalation. The packaging device or dispenser may be accompanied by instructions for administration. Compositions comprising a compound of the present invention formulated in a compatible pharmaceutical carrier may also be placed in an appropriate container and labeled for treatment of an indicated condition.

### Indications

The compounds of the present invention are useful in modulating CRTH2-mediated activity and, based on the target rationale and in vitro potency, one would expect they would be useful as anti-inflammatory agents for the treatment, amelioration or prevention of inflammatory diseases and of complications arising therefrom.

Inflammation of tissues and organs occurs in a wide range of disorders and diseases and in certain variations results from activation of the cytokine family of receptors. Exemplary inflammatory disorders associated with activation of CRTH2 include, in a non-limiting manner, skin disorders, respiratory disorders, and other disorders with an allergic component. These disorders are treated or prevented by modulation of CRTH2 activity, for example, by administration of an inhibitor according to the present invention.

Exemplary skin disorders include dermatitis, cutaneous eosinophilias, Lichen planus, urticaria, psoriasis, pruritus, angiodermas, chronic skin ulcers, conjunctivitis, vasculitides, or erythemas. Examples of respiratory disorders include asthma, rhinitis, chronic obstructive pulmonary disease, bronchitis, nasal polyposis, nasal congestion, farmer's lung, fibroid lung and cough. Other exemplary diseases affected by CRTH2 include osteoarthritis, rheumatoid arthritis, corneal ulcers, uveitis, pain and inflammatory bowel disease.

According to the present invention, the CRTH2 inhibitors may be administered prophylactically, i.e., prior to onset of acute allergic reaction, or they may be administered after onset of the reaction, or at both times.

The following examples will further describe the invention, and are used for the purposes of illustration only, and should not be considered as limiting the invention being disclosed.

### EXAMPLES

The following abbreviations and terms have the indicated meaning throughout:

Ac=acetyl

Boc=tert-butoxycarbonyl

BSA=bovine serum albumin

Bu=butyl

BuOH=butanol

CDCl₃=Deuterated chloroform

CD₃OD=Deuterated methanol

CHO=Chinese hamster ovary

δ=NMR chemical shift referenced to tetramethylsilane

DCM=dichloromethane=methylene chloride=CH₂Cl₂

DCE=1,2-dichloroethane

DEAD=diethyl azodicarboxylate

DHK-PGD₂=13,14-dihydro-15-keto-prostaglandin D2

DIC=diisopropylcarbodiimide

DIEA=N,N-diisopropylethyl amine

DMF=N,N-dimethylformamide

DMSO=dimethyl sulfoxide

EA (EtOAc)=Ethyl Acetate

EDC=N-(3-Dimethylaminopropyl)-N′)ethylcarbodiimide

EtOH=ethanol

GC=gas chromatography

GDP=Guanosine diphosphate

h=hours

HEPES=(4-(2-hydroxyethyl)-1-piperazineethanesulfonic acid)

HOAc=acetic acid

HOBt=hydroxybenzotriazole

m-=meta

Me=methyl

MeOH=methanol=CH₃OH

MS=mass spectrometry

min=minutes

n=normal

NMR=Nuclear Magnetic Resonance

Na(OAc)₃BH=sodium triacetoxy borohydride

o-=ortho

p-=para

Pd(dppf)₂Cl₂=dichloro[1,1′-bis(diphenylphosphinoferrocene]palladium

PGD2=prostaglandin D2

Ph=phenyl

PhOH=phenol

RT=room temperature

sat'd=saturated

s-=secondary

SPA=scintillation proximity assay

t-=tertiary

TBDMS=t-butyldimethylsilyl

TFA=trifluoroacetic acid

THF=tetrahydrofuran

TMOF=trimethyl orthoformate

TMS=trimethylsilyl

tosyl=p-toluenesulfonyl

Trt=triphenylmethyl

Examples below describe syntheses of certain precursors and intermediates of the invention. Compounds of formula I were synthesized by means of conventional organic synthesis executable by those skilled in the art. The illustration of examples, but not the limitation, of the synthesis of compounds of formula I is detailed herein below:

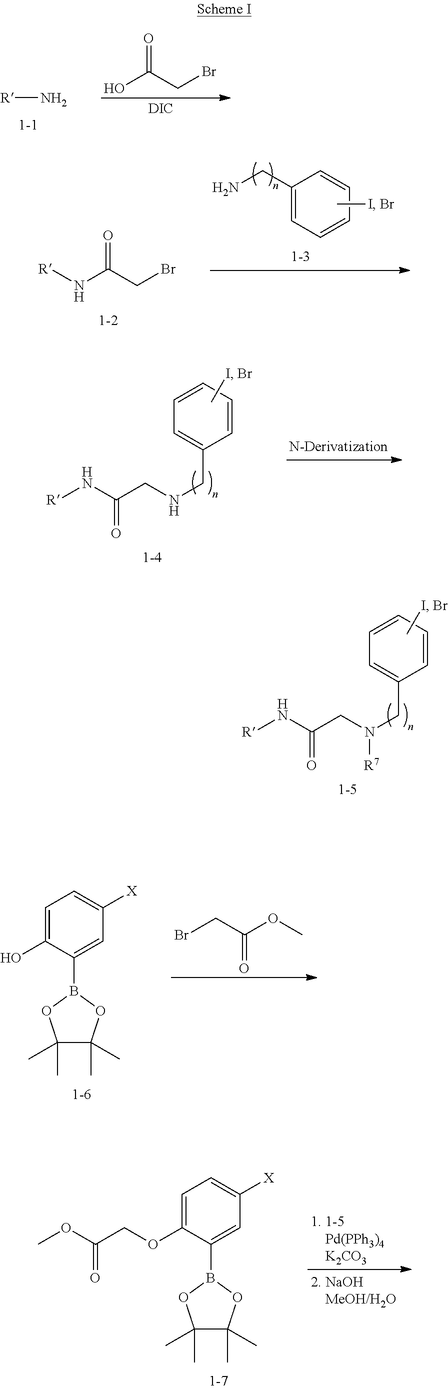

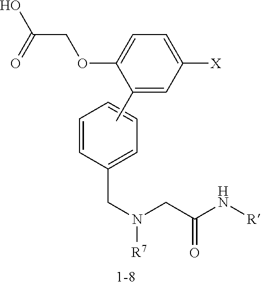

As shown in Scheme 1, treatment of amine 1-1 with 2-bromoacetic acid in the presence DIC generates 2-bromoacetic amide 1-2. Nucleophilic displacement of bromide 1-2 with amine 1-3 affords acetamide 1-4. Derivatization of intermediate 1-4, (for example with an activated carboxylic acid, acid chloride, sulfonyl chloride, isocyanate, or chloroformate) yields amino acetamide intermediate 1-5. Independently, reaction of the 4-substituted-2-(4,4,5,5-tetramethyl-1,3,2-dioxaborolan-2-yl)phenol 1-6 with methyl bromoacetate in the presence of K₂CO₃ yields the boronate ester 1-7. Subsequent Suzuki coupling of 1-5 and boronate ester 1-7 followed by deprotection in an aqueous NaOH methanol solution provides phenoxyacetic acid 1-8 of the invention.

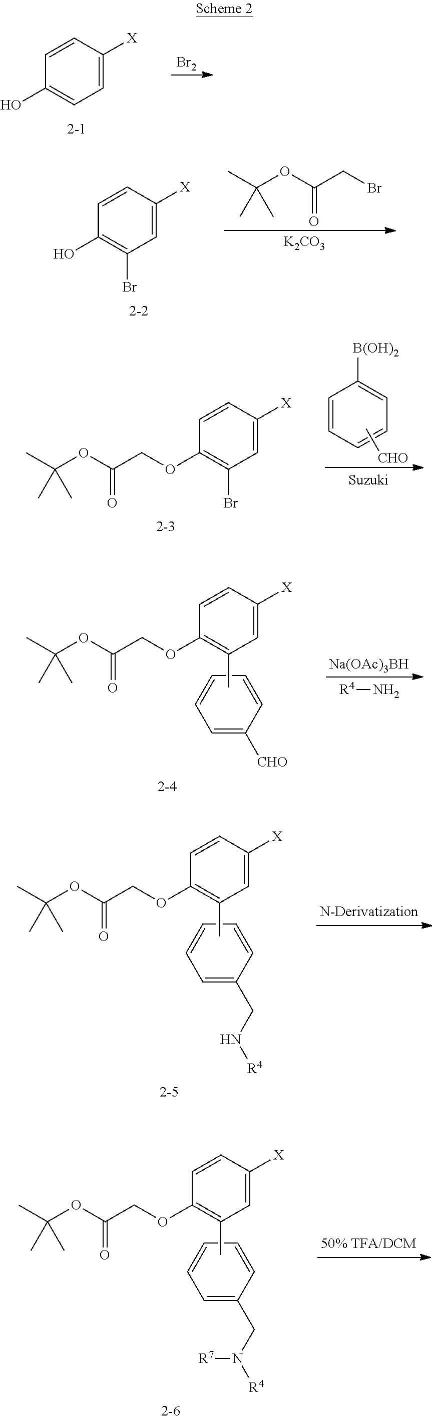

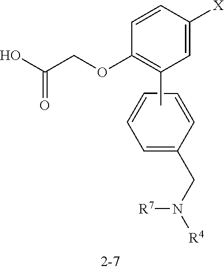

Another method for preparing compounds of the invention and intermediates thereof is illustrated in Scheme 2. As shown, bromination of a substituted phenol produces the desired ortho-substituted derivative 2-2 which can be alkylated with tert-butyl 2-bromoacetate in the presence of a suitable base such as potassium carbonate to yield intermediate 2-3. Palladium-catalyzed coupling with a formyl boronic acid gives the biaryl aldehyde 2-4. Reductive alkylation with a primary amine generates secondary amine 2-5 which can be subsequently N-derivatized by a number of methods. The resultant intermediate 2-6 is then deprotected under acidic conditions to produce the carboxylic acid compounds 2-7 of the invention.

### Example 1

### (Intermediate) Butyl 3-iodobenzyl(2-(butylamino)-2-oxoethyl)carbamate (1)

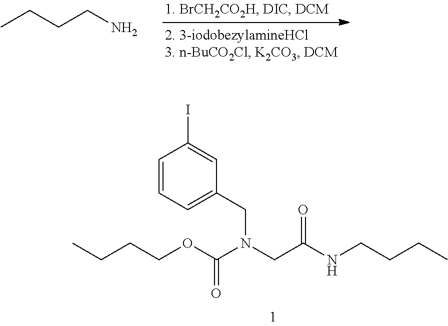

To a solution of n-butylamine (0.35 mL, 3.5 mmol, 1 eq), 1,3-diisopropylcarbodiimide (DIC) (0.55 mL, 3.5 mmol, 1 eq) in anhydrous DCM (40 mL) at 0° C. was added bromoacetic acid (0.49 g, 3.5 mmol, 1 eq). The mixture was allowed to warm to RT and stirred for 2 h. After the reaction was complete as determined by LC/MS, n-butyl 3-iodobenzylamine (2.45 g, 10.5 mmol, 3 eq) was added and the reaction was stirred at RT for 1 h. After LC/MS showed the disappearance of starting material, n-butyl chloroformate (1 mL, 7.86 mmol, 2.25 eq) and K₂CO₃ (1.1 g, 7.96 mmol, 2.3 eq) were added directly to the reaction mixture. The reaction was further stirred at RT for 1 h, poured into 10 mL of H₂O, and extracted with DCM (3×20 mL). The combined organics were washed with brine (10 mL), dried (Na₂SO₄), and concentrated in vacuo. The residue was purified by silica gel chromatography eluting with a gradient of 100% hexanes to 40% EtOAc/60% hexanes to give 0.50 g (33%) of butyl 3-iodobenzyl(2-(butylamino)-2-oxoethyl)carbamate (1) as a white solid. ¹HNMR (CDCl₃, 300 MHz): δ 7.62 (m, 2H), 7.22 (bs, 1H), 7.06 (t, J=8.0 Hz, 1H), 5.91 (bd, 1H), 4.48 (m, 2H). 4.17 (t, J=6.6 Hz, 2H), 3.83 (s, 2H), 3.25 (m, 2H), 1.63 (m, 2H), 1.38 (m, 6H), 0.92 (m, 6H). MS (ESI): MH⁺=447.2. HPLC (ZQ) t_{R}=7.52 min.

### Example 2

### Methyl 2-(2-(4,4,5,5-tetramethyl-1,3,2-dioxaborolan-2-yl)phenoxy)acetate (2)

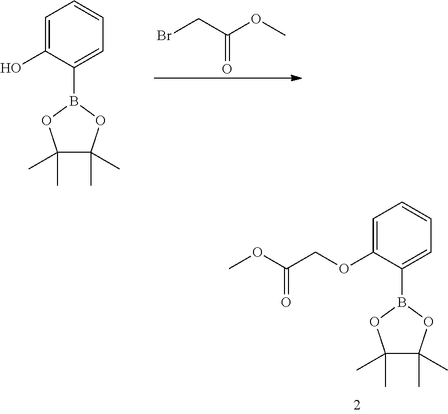

To a solution of 2-(4,4,5,5-tetramethyl-1,3,2-dioxaborolan-2-yl)phenol (2.53 mmol, 1 eq) in 5 mL of DMF was added methyl bromoacetate (6.34 mmol, 2.5 eq), and the K₂CO₃ (10.1 mmol, 4 eq). The reaction mixture was heated to 70° C. for 16 h, cooled to RT and the solvent was removed in vacuo. The mixture was then partitioned between H₂O (10 mL) and EtOAc (10 mL). The organic layer was separated and the aqueous layer was extracted with EtOAc (3×10 mL). The combined organic layers were washed with brine, dried (MgSO₄) and concentrated to give brown oil. The residue was purified by silica gel chromatography eluting with a linear gradient of 0% EtOAc/hexanes to 20% EtOAc/hexanes to give 0.57 g (76%) of methyl 2-(2-(4,4,5,5-tetramethyl-1,3,2-dioxaborolan-2-yl)phenoxy)acetate (2) as a clear oil. ¹HNMR (CDCl₃, 400 MHz): δ 7.69 (dd, J=8.0, 4.0 Hz, 1H), 7.37 (m, 1H), 7.01 (dd, J=8.0 Hz, 1H), 6.81 (d, J=8.0 Hz, 1H), 4.65 (s, 2H), 3.79 (s, 3H), 1.36 (s, 12H).

### Example 3

### Methyl 2-(2-(4,4,5,5-tetramethyl-1,3,2-dioxaborolan-2-yl)4(trifluoromethyl)phenoxy)acetate (3)

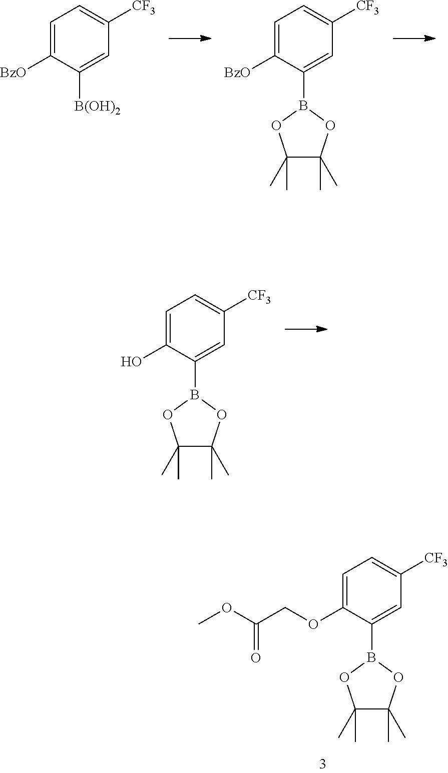 [0148] (a) 2-(4,4,5,5-tetramethyl-1,3,2-dioxaborolan-2-yl)-4-(trifluoromethyl)phenol: To a RB flask fitted with a reflux condenser and Dean Stark trap was added 2-(benzyloxy)-5-(trifluoromethyl)phenylboronic acid (0.8 g, 2.7 mmol, 1 eq) and pinacol (0.4 g, 3.37 mmol, 1.25 eq) in toluene. The reaction mixture was refluxed for 2 days and TLC showed no starting material left. The solvent was removed and the residue was dissolved in EtOH (60 mL) and 10% palladium on carbon (0.16 g, catalyst) was added. The mixture was hydrogenated under a hydrogen balloon at RT for 24 h. The reaction mixture was filtered through Celite and the filtrate was concentrated in vacuo to give 0.68 g (87%) of 2-(4,4,5,5-tetramethyl-1,3,2-dioxaborolan-2-yl)-4-(trifluoromethyl)phenol. ¹H NMR (CDCl₃, 300 MHz): δ 8.15 (s, 1H), 7.88 (d, J=1.5 Hz, 1H), 7.59 (dd, J=2.1, 8.7 Hz, 1H), 6.94 (d, J=8.7 Hz, 1H), 1.38 (s, 12H). ¹⁹F NMR (CDCl₃, 300 MHz) δ−61.81
(b) Following a similar procedure as in Example 5, treatment of 2-(4,4,5,5-tetramethyl-1,3,2-dioxaborolan-2-yl)-4-(trifluoromethyl)phenol with methyl bromoacetate (2 eq) and K₂CO₃ (4 eq) yielded methyl 2-(2-(4,4,5,5-tetramethyl-1,3,2-dioxaborolan-2-yl)-4-(trifluoromethyl)phenoxy)acetate (3) as a white solid (33% yield). ¹HNMR (CDCl₃, 300 MHz): δ 7.91 (d, J=2.1 Hz, 1H), 7.60 (dd, J=8.7, 2.4 Hz, 1H), 6.80 (d, J=9.0 Hz, 1H), 4.69 (s, 2H), 3.78 (s, 3H), 1.35 (s, 12H).

### Example 4

### Methyl 2-(4-methyl-2-(4,4,5,5-tetramethyl-1,3,2-dioxaborolan-2-yl)phenoxy)acetate (4)

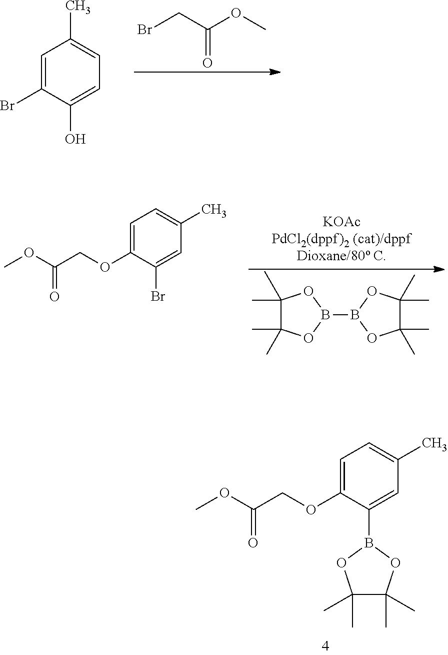 [0151] (a) To 2-bromo-4-methylphenol (1.16 g, 6.2 mmol, 1 eq) in 15 mL of DMF was added methyl bromoacetate (1.17 mL, 12.4 mmol, 2 eq), and K₂CO₃ (3.42 g, 24.8 mmol, 4 eq). After following the procedure outlined in example 5, 1.38 g (86%) of methyl 2-(2-bromo-4-methylphenoxy)acetate was obtained as a white solid. ¹H NMR (CDCl₃, 300 MHz): δ 7.38 (d, J=1.8 Hz, 1H), 7.26 (dd, J=8.4, 1.5 Hz, 1H), 6.21 (d, J=8.4 Hz, 1H), 4.68 (s, 2H), 3.79 (s, 3H), 2.27 (s, 3H).
(b) The synthesis was adapted from the procedure of Hunter, L.; Hutton, C. A. Aust. J. Chem., 2003, 56(11), 1095-98. Thus, to a 10 mL dioxane solution of methyl 2-(2-bromo-4-methylphenoxy)acetate (0.46 g, 1.79 mmol, 1 eq) was added potassium acetate (0.53 g, 5.4 mmol, 3 eq), diboronpicacolester (0.7 g, 2.7 mmol, 1.5 eq), Pd(dppf)Cl₂ (0.04 g, 0.05 mmol, 3 mol %), and 1,1′-bis(diphenylphosphino)ferrocene (0.03 g, 0.05 mmol, 3 mol %). After heating at 80° C. for 16 h, the solvent was evaporated under reduced pressure. The residue was dissolved in EtOAc-H₂O, filtered through Celite, and the organic layer was separated. The aqueous layer was extracted with EtOAc (3×10 mL), the combined organic layers were dried (MgSO₄), and the solution concentrated to give brown oil. The residue was purified by silica gel column chromatography eluted with a linear gradient of 0% EtOAc/hexanes to 10% EtOAc/hexanes which gave 0.06 g (12%) of methyl 2-(4-methyl-2-(4,4,5,5-tetramethyl-1,3,2-dioxaborolan-2-yl)phenoxy)acetate (4) as an oil. ¹HNMR (CDCl₃, 300 MHz): δ 7.49 (d, J=2.1 Hz, 1H), 7.15 (dd, J=8.4, 2.1 Hz, 1H), 6.73 (d, J=8.4 Hz, 1H), 4.62 (s, 2H), 3.78 (S, 3H), 2.27 (s, 3H), 1.36 (s, 12H).

### Example 5

### Methyl 2-(4-fluoro-2-(4,4,5,5-tetramethyl-1,3,2-dioxaborolan-2-yl)phenoxy)acetate (5)

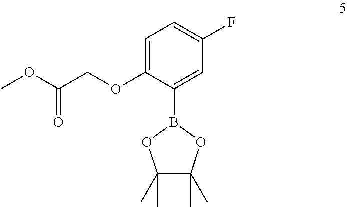

Following a similar procedure as in Example 7 except replacing 2-bromo-4-methylphenol with 2-bromo-4-fluorophenol, methyl 2-(4-fluoro-2-(4,4,5,5-tetramethyl-1,3,2-dioxaborolan-2-yl)phenoxy)acetate was obtained as a clear oil (0.08 g, 12%). ¹HNMR (CDCl₃, 300 MHz): δ 7.36 (dd, J=8.4, 3.3 Hz, 1H), 7.03 (ddd, J=3.3, 9.0, 7.8 Hz, 1H), 6.73 (dd, J=4.2, 9.0 Hz, 1H), 4.61 (s, 2H), 3.79 (s, 3H), 1.35 (s, 12H). ¹⁹FNMR (CDCl₃, 300 MHz): δ−122.78, d, J=9.6 Hz.

### Example 6

### {[3′-({(butoxy carbonyl)[2-(butylamino)-2-oxoethyl]amino}methyl)biphenyl-2-yl]oxy}acetic acid (6)

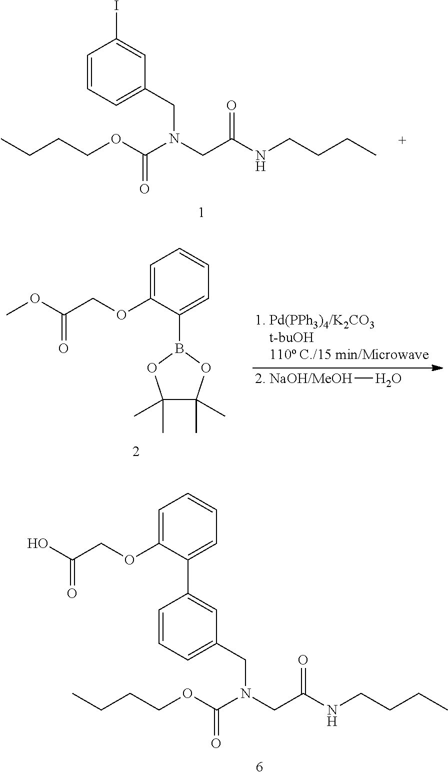

In a Smith process (1-5 mL) vial equipped with a stir bar was added a solution of butyl 3-iodobenzyl(2-(butylamino)-2-oxoethyl)carbamate 1 (0.06 g, 0.13 mmol) in t-BuOH (1 mL), and methyl 2-(2-(4,4,5,5-tetramethyl-1,3,2-dioxaborolan-2-yl)phenoxy)acetate (0.08 g, 0.27 mmol, 2 eq). To this solution mixture, Pd (PPh₃)₄ (0.0014 g, 9 mol %), and K₂CO₃ (1M in H₂O, 0.26 mL, 0.26 mmol, 2 eq) were added. The reaction vessel was sealed and heated using microwave irradiation to 110° C. for 15 min. After cooling, the solvent was evaporated under reduced pressure and the residue was dissolved in EtOAc-H₂O. The organic layer was separated and the aqueous layer was extracted with EtOAc (3×2 mL). The combined organic layers were dried (MgSO₄) and concentrated to give a brown oil which was purified by silica gel chromatography using a linear gradient of 30% EtOAc/hexanes to 50% EtOAc/hexanes to yield (0.02 g, 29%) of phenoxyacetic ester as yellow oil. The ester was dissolved in 2 mL of methanol-H₂O with 2 eq of NaOH added and stirred at RT for 24 h. The solvent was evaporated and the residue was purified by reverse phase preparative HPLC to give 0.014 g (73%) of 6 as a white solid. ¹HNMR (CDCl₃, 300 MHz): δ 7.74 (m, 1H), 7.40 (m, 2H), 7.37-7.27 (m, 2H), 7.18-7.06 (m, 3H), 6.90 (d, J=7.2 Hz, 1H), 4.55 (s, 2H), 4.53 (s, 2H), 4.20 (bs, 2H), 3.92 (bs, 2H), 3.16 (m, 2H), 1.65 (bs, 2H), 1.5-1.14 (m, 6H), 0.92-0.85 (m, 6H). MS (ESI): MH⁺=471.1. HPLC (ZQ) t_{R}=6.73 min.

### Example 7

### {[3′-({(butoxy carbonyl)[2-(butylamino)-2-oxoethyl]amino}methyl)-5-fluorobiphenyl-2-yl]oxy}acetic acid (7)

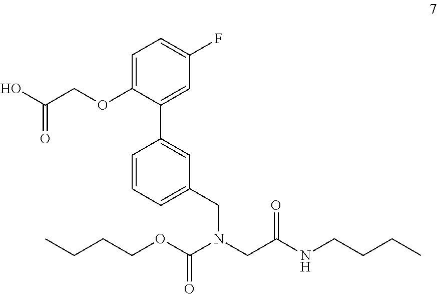

Following the procedure as in example 6 except starting with methyl 2-(4-fluoro-2-(4,4,5,5-tetramethyl-1,3,2-dioxaborolan-2-yl)phenoxy)acetate and 0.11 g of butyl 3-iodobenzyl(2-(butylamino)-2-oxoethyl)carbamate 1, {[3′-({(butoxy carbonyl)[2-(butylamino)-2-oxoethyl]amino}methyl)-5-fluorobiphenyl-2-yl]oxy}acetic acid (7) was obtained as a white solid (0.08 g; 68%). ¹HNMR (CD₃OD, 300 MHz): δ 7.52 (m, 2H), 7.39 (dd, J=7.5, 7.8 Hz, 1H), 7.26 (m, 1H), 7.08 (dd, J=9, 2.4 Hz, 1H), 7.05-6.96 (m, 2H), 4.60 (m, 4H), 4.15 (m, 2H), 3.90 (m, 2H), 3.19 (m, 2H), 1.63 (m, 2H), 1.58-1.26 (m, 6H), 0.93 (m, 6H). ¹⁹FNMR (CD₃OD, 300 MHz): −124.99. MS (ESI): MH⁺=489.0. HPLC (LCQ) t_{R}=7.15 min.

### Example 8

### {[3′-({(butoxy carbonyl)[2-(butylamino)-2-oxoethyl]amino}methyl)-5-methylbiphenyl-2-yl]oxy}acetic acid (8)

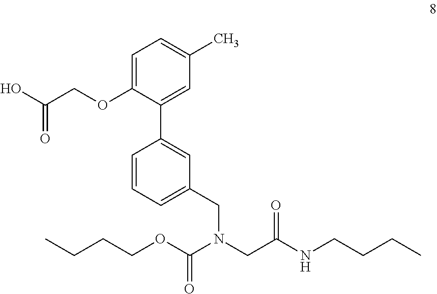

Starting with butyl 3-iodobenzyl(2-(butylamino)-2-oxoethyl)carbamate 1 (0.06 g, 0.14 mmol) and methyl 2-(4-methyl-2-(4,4,5,5-tetramethyl-1,3,2-dioxaborolan-2-yl)phenoxy)acetate, and following the procedure outlined in example 6, {[3′-({(butoxy carbonyl)[2-(butylamino)-2-oxoethyl]amino}methyl)-5-methylbiphenyl-2-yl]oxy}acetic acid (8) was obtained as a white solid (9.2 mg; 0.019 mmol; 14%). ¹HNMR (CD₃OD, 300 MHz): δ 7.50 (s, 1H), 7.47 (d, J=7.8 Hz, 2H), 7.36 (dd, J=7.5, 7.8 Hz, 1H), 7.19 (m, 1H), 7.10 (m, 2H), 6.86 (d, J=8.1 Hz, 1H), 4.58 (s, 4H), 4.14 (m, 2H), 3.90 (m, 2H), 3.14 (t, J=6.6 Hz, 2H), 2.31 (s, 3H), 1.63 (m, 2H), 1.48-1.24 (m, 6H), 0.91 (m, 6H). MS (ESI): MH⁺=484.0. HPLC (ZQ) t_{R}=6.98 min.

### Example 9

### {[3′-({(butoxy carbonyl)[2-(butylamino)-2-oxoethyl]amino}methyl)-5-(trifluoromethyl)biphenyl-2-yl]oxy}acetic acid (9)

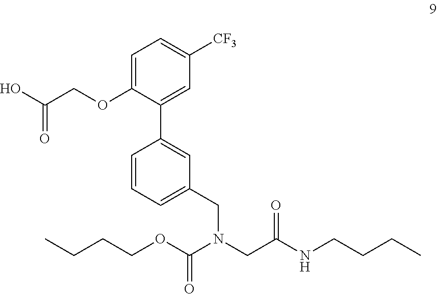

Starting with butyl 3-iodobenzyl(2-(butylamino)-2-oxoethyl)carbamate 1 (0.09 g, 0.20 mmol) and methyl 2-(2-(4,4,5,5-tetramethyl-1,3,2-dioxaborolan-2-yl)4(trifluoromethyl)phenoxy)acetate, and following the procedure outlined in example 6, {[3′-({(butoxy carbonyl)[2-(butylamino)-2-oxoethyl]amino}methyl)-5-(trifluoromethyl)biphenyl-2-yl]oxy}acetic acid (9) was obtained as a white solid 76 mg (71%). ¹HNMR (CD₃OD, 300 MHz): δ 7.60-7.44 (m, 4H), 7.38 (dd, J=7.8 Hz, 1H), 7.26 (m, 1H), 7.08 (J=8.4 Hz, 1H), 4.58 (s, 2H), 4.53 (s, 2H), 4.13 (m, 2H), 3.91 (m, 2H), 3.15 (bs, 2H), 1.61 (m, 2H), 1.50-1.22 (m, 6H), 0.90 (m, 6H). ¹⁹FNMR (CD₃OD, 300 MHz): −63.34. MS (ESI): MH⁺=538.0. HPLC (ZQ) t_{R}=7.25 min.

### Example 10

### {[3′-({(butoxy carbonyl)[2-(butylamino)-2-oxoethyl]amino}methyl)-5-chlorobiphenyl-2-yl]oxy}acetic acid (11)

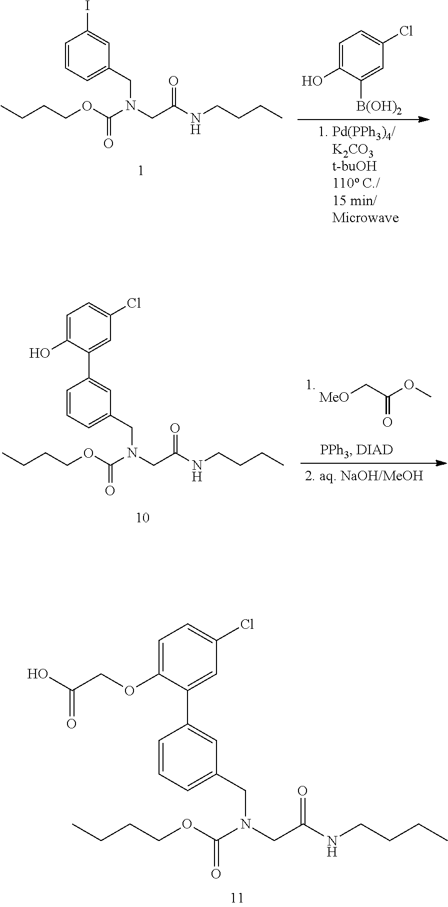 [0164] (a) Following a similar procedure as in example 6, starting with butyl 3-iodobenzyl(2-(butylamino)-2-oxoethyl)carbamate 1 (0.20 g, 0.45 mmol) and 5-chloro-2-hydroxyphenylboronic acid, phenol 10 was obtained as a yellow oil 0.12 g (60%). ¹HNMR (CDCl₃, 300 MHz): δ 7.46-7.33 (m, 3H), 7.23 (bs, 1H), 7.20-7.13 (m, 2H), 6.91 (d, J=8.4 Hz, 1H), 6.50-5.64 (bs, 1H), 4.56 (s, 2H), 4.18 (m, 2H), 3.86 (s, 2H), 3.07 (m, 2H), 1.67 (m, 2H), 1.46-1.14 (m, 6H), 0.93 (t, J=7.2 Hz, 3H), 0.86 (t, J=7.2 Hz, 3H). MS (ESI): MH⁺=447.3. HPLC (ZQ) t_{R}=7.35 min.
(b) According to the procedure of George, G. et al, Tetrahedron Lett., 1998, 39, 8751-8754, to a solution of phenol 10 (0.04 g, 0.09 mmol, 1 eq) in DCM was added methylglycolate (0.015 mL, 0.19 mmol, 2 eq), followed by polymer-bound triphenylphosphine (2 eq, 3 mmol/g resin). After stirring 30 min, the reaction mixture was cooled to 0° C., DIAD (0.04 mL, 0.19 mmol, 2 eq) was added, and the mixture was stirred under argon at RT for 48 h. The resin was removed by filtration and washed with DCM (2×). The filtrate was washed with 5% KOH, 5% HCl, and brine, dried (MgSO₄), filtered, and concentrated in vacuo to give the crude phenoxy ester. Deprotection with 2 eq of NaOH in methanol/H₂O (1 mL) for 3 days yielded {[3′-({(butoxy carbonyl)[2-(butylamino)-2-oxoethyl]amino}methyl)-5-chlorobiphenyl-2-yl]oxy}acetic acid (11) (0.073 g, 16%) of the invention. ¹HNMR (CD₃OD, 300 MHz): δ 7.53-7.45 (m, 2H), 7.38 (dd, J=7.5 Hz, 1H), 7.32-7.20 (m, 3H), 6.97 (J=9.0 Hz, 1H), 4.66 (s, 2H), 4.58 (s, 2H), 4.13 (m, 2H), 3.88 (bs, 2H), 3.15 (t, J=6.9 Hz, 2H), 1.62 (m, 2H), 1.48-1.22 (m, 6H), 0.90 (m, 6H). MS (ESI): MH⁺=504.0. HPLC (ZQ) t_{R}=7.13 min.

### Example 11

### N-(3-iodobenzyl)-N-(2-(butylamino)-2-oxoethyl)phenylacetamide (12)

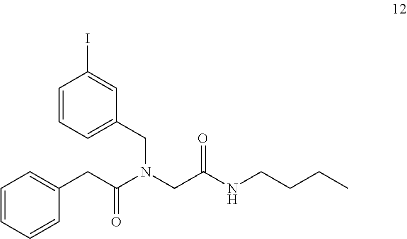

Following a similar procedure as in Example 1 using phenylacetyl chloride in place of n-butyl chloroformate provided N-(3-iodobenzyl)-N-(2-(butylamino)-2-oxoethyl)phenylacetamide (12) as a white solid. ¹H NMR (CDCl₃, 300 MHz) δ 7.66-7.56 (m, 1H), 7.38-7.18 (m, 6H), 7.01-6.98 (m, 2H), 4.56 (m, 2H), 3.89 (m, 2H), 3.75 (m, 2H), 3.24-3.03 (m, 2H), 1.48-1.18 (m, 4H), 0.9 (t, J=7.5 Hz, 3H).

### Example 12

### {[3′-{[[2-(butylamino)-2-oxoethyl](phenylacetyl)amino]methyl}-biphenyl-2-yl]oxy}acetic acid (13)

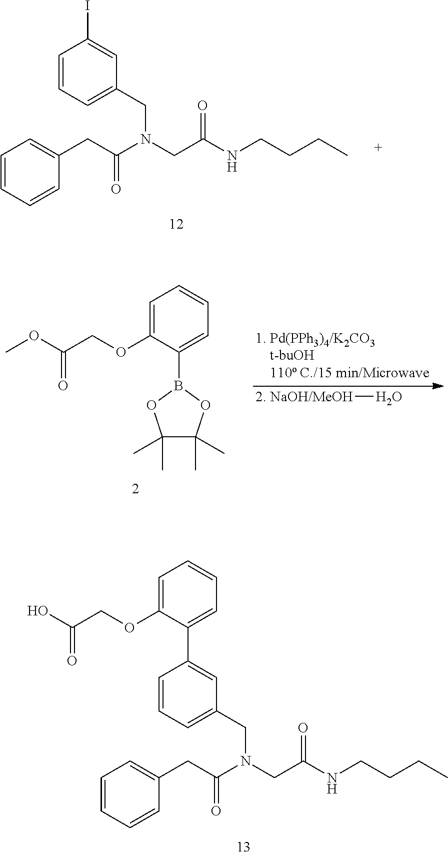

Following a similar procedure as in example 6 except replacing butyl 3-iodobenzyl(2-(butylamino)-2-oxoethyl)carbamate with N-(3-iodobenzyl)-N-(2-(butylamino)-2-oxoethyl)phenylacetamide, 0.05 g (76%) of {[3′-{[[2-(butylamino)-2-oxoethyl](phenylacetyl)amino]methyl}-biphenyl-2-yl}oxy]acetic acid (13) was obtained as a white solid. ¹HNMR (CD₃OD, 300 MHz): δ 7.53-7.38 (m, 3H), 7.35-7.19 (m, 7H), 7.12-7.03 (m, 2H), 6.93 (d, J=8.1 Hz, 1H), 4.70 (m, 2H), 4.57 (m, 2H), 3.99 (m, 2H), 3.83 (m, 2H), 3.18-3.06 (m, 2H), 1.46-1.20 (m, 4H), 0.88 (t, J=7.2 Hz, 3H). MS (ESI): MH⁺=488.0. HPLC (ZQ) t_{R}=6.42 min.

### Example 13

### tert-butyl 2-(2-bromo-4-(trifluoromethyl)phenoxy)acetate (15)

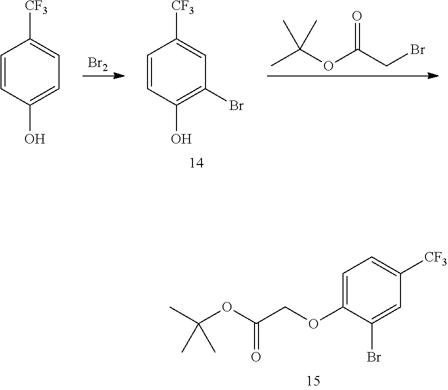 [0171] a) 4.8 ml of Bromine (92.6 mmol, 1 eq.) in 50 ml of DCM was added dropwise to a solution of 15 g of 4-(trifluoromethyl)phenol (92.6 mmol, 1 eq.) in 200 ml of DCM at rt. The mixture was stirred overnight and then washed with aqueous Na₂SO₃ and brine, dried, and evaporated in vacuo to give 20 g (90%) 2-bromo-4-(trifluoromethyl)phenol (14) exhibiting 90% purity by HPLC.
b) To a solution of 20 g of 14 (83 mmol, 1 eq.) in 250 ml of DMF was added 24 g of tert-butyl bromoacetate (124 mmol, 1.5 eq.) and 54 g of Cs₂CO₃ (166 mmol, 2 eq). The reaction mixture was stirred overnight at RT, filtered, and the filtrate was concentrated to afford crude material which was purified by flash chromatography over silica gel (200-300 m, eluting with 8:1 petroleum ether:ethyl acetate) to give 27 g (92%) of pure tert-butyl 2-(2-bromo-4-(trifluoromethyl)phenoxy)acetate (15).

### Example 14

### tert-butyl 2-(2-3-formylphenyl-4-(trifluoromethyl)phenoxy)acetate (16)

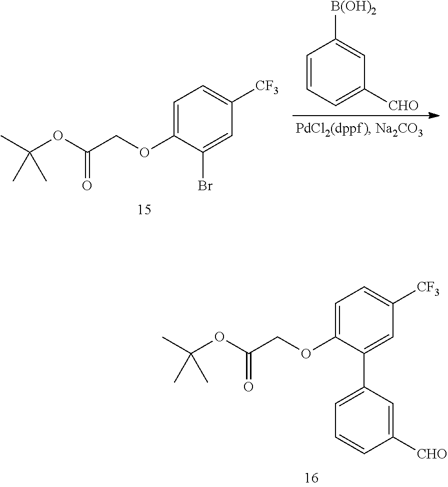

To a solution of 1 g of 15 (2.8 mmol, 1 eq.) in 50 ml of THF\toluene\H₂O (2:2:1) was added 465 mg of 3-formylphenylboronic acid (3.1 mmol, 1.1 eq.), 115 mg of PdCl₂(dppf) (0.14 mmol, 0.05 eq.) and 600 mg of Na₂CO₃ (5.6 mmol, 2 eq.). The reaction mixture was heated to 80° C. overnight under nitrogen. After extraction with DCM, the organic layer was concentrated to give crude material which was purified by flash chromatography over silica gel (200-300 m, eluting with 35:1 petroleum ether:ethyl acetate) to yield 0.8 g (75%) of (16). ¹HNMR (CDCl₃, 300 MHz): δ 10.07 (s, 1H), 8.09 (t, J=1.5 Hz, 1H), 7.88 (dd, J=1.5 Hz, 2H), 7.60 (m, 3H), 6.89 (d, J=8.4 Hz, 1H), 4.58 (s, 2H), 1.46 (s, 9H).

### Example 15

### {[3′-{[(butoxycarbonyl)(methyl)amino]methyl}-5-(trifluoromethyl)biphenyl-2-yl]oxy}acetic acid (17)

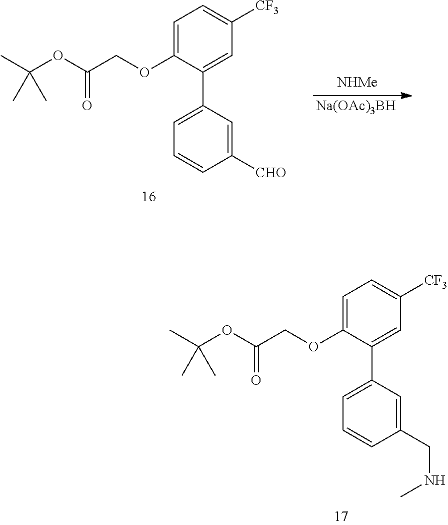 [0176] b) To a solution of aldehyde 16 (0.04 g, 0.1 mmol, 1 eq) was added methylamine (0.1 mL, 0.2 mmol, 2 eq, 2M solution in THF) in DCE (1 mL) and the reaction mixture was stirred for 3 h at RT. NaBH(OAc)₃ (0.04 g, 0.2 mmol, 2 eq) was added and the reaction mixture was stirred for 16 h and quenched with aqueous Na₂CO₃. After extraction with CHCl₃ (4×2 mL), the combined organic layers were dried (MgSO₄) and concentrated to give crude amine 17 which was used for the next step without further purification.

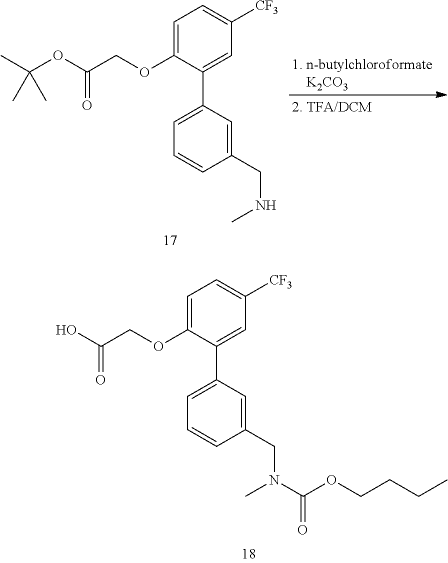

c) To a solution of amine 17 (1 eq) in anhydrous DCM (1 mL) was added K₂CO₃ (0.06 g, 0.41 mmol, 2 eq) and n-butyl chloroformate (0.03 mL, 0.21 mmol, 2 eq). The reaction was stirred at RT for 3 h. After LC/MS indicated reaction completion, the solvent was evaporated and the residue was purified by reverse phase preparative HPLC to give 35 mg (76%) of ester. The t-butyl ester was deprotected by treatment with 50% TFA/DCM for 2 h. The solvent was evaporated and the residue was purified by reverse phase preparative HPLC to give 26 mg (76%) of 18 as a white solid. ¹HNMR (CDCl₃, 300 MHz): δ, 7.62-7.53 (m, 2H), 7.50-7.37 (m, 3H), 7.23 (bs, 1H), 6.94 (d, J=8.4 Hz, 1H), 4.68 (s, 2H), 4.52 (s, 2H), 4.13 (t, J=6.6 Hz, 2H), 2.91 (bs, 3H), 1.61 (bs, 2H), 1.35 (bs, 2H), 0.90 (m, 3H). ¹⁹FNMR (CDCl₃, 300 MHz): −62.04. MS (ESI): MH⁺=439.0. HPLC (ZQ) t_{R}=7.5 min.

### Example 16

### {[3′-{[(butoxycarbonyl)(butyl)amino]methyl}-5-(trifluoromethyl)biphenyl-2-yl]oxy}acetic acid (19)

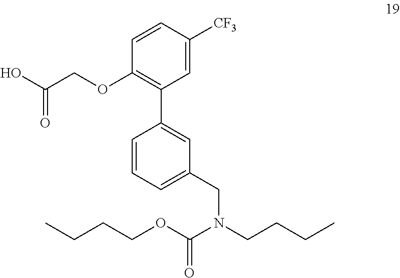

Following the procedure outlined in Example 15 except replacing methylamine with n-butylamine yielded compound 19 (15 mg, 25%) as a white solid. ¹HNMR (CD₃OD, 300 MHz): δ 7.60 (dd, J=8.7, 2.1 Hz, 1H), 7.56-7.45 (m, 3H), 7.39 (dd, J=7.5 Hz, 8.4 Hz, 1H), 7.25 (bs, 1H), 7.12 (d, J=8.7 Hz, 1H), 4.76 (s, 2H), 4.53 (s, 2H), 4.10 (bs, 2H), 3.29 (bs, 2H), 1.72-1.37 (m, 5H), 1.37-1.20 (m, 3H), 0.90 (m, 6H). ¹⁹FNMR (CD₃OD, 300 MHz): −59.47. MS (ESI): MH⁺=482.1. HPLC (ZQ) t_{R}=9.50 min.

### Example 17

### {[3′-({(butoxycarbonyl) [2-(methylamino)-2-oxoethyl]amino}methyl)-5-(trifluoromethyl)biphenyl-2-yl]oxy}acetic acid (20)

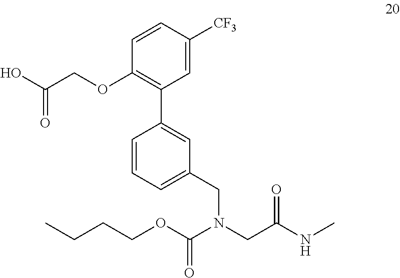 [0181] (a) 2-(tert-butoxycarbonyl)glycine (1 g, 5.7 mmol, 1 eq), methyl amine (2.9 mL, 5.7 mmol, 1 eq), and EDC (1.1 g, 5.7 mmol, 1 eq) were stirred in DCM (10 mL) at RT for 16 h. The reaction was washed with saturated aqueous NaHCO₃, brine, dried (Na₂SO₄), and concentrated in vacuo to afford 0.3 g (28%) of the tert-butyl 2-(methylamino)-2-oxoethylcarbamate. The crude tert-butyl 2-(methylamino)-2-oxoethylcarbamate was treated with 30% TFA/DCM (3 mL) at RT for 3 h. After the solvent was evaporated under reduce pressure, glycine methylacetamide was obtained as a light yellow residue (0.27 g, 90%) as a TFA salt.
(b) Following a similar procedure as in Example 15 except replacing methylamine with tert-butyl 2-(methylamino)-2-oxoethylcarbamate yielded {[3′-({(butoxycarbonyl) [2-(methylamino)-2-oxo ethyl]amino}methyl)-5-(trifluoromethyl)biphenyl-2-yl]oxy}acetic acid (20) (0.01 g, 20%) as a white solid. ¹HNMR (CD₃OD, 300 MHz): δ 7.60 (d, J=8.7 Hz, 1H), 7.57-7.46 (m, 3H), 7.41 (dd, J=7.5 Hz, 7.8 Hz, 1H), 7.26 (bs, 1H), 7.13 (d, J=8.4 Hz, 1H), 4.78 (s, 2H), 4.59 (s, 2H), 4.13 (t, J=6.3 Hz, 2H), 3.88 (m, 2H), 2.70 (s, 3H), 1.62 (m, 2H), 1.36 (m, 2H), 0.90 (m, 3H). ¹⁹FNMR (CD₃OD, 300 MHz): −77.62. MS (ESI): MH⁺=497.1. HPLC (ZQ) t_{R}=6.55 min.

### Example 18

### {[3′-({(acetyl)[2-(methylamino)-2-oxoethyl]amino}methyl)-5-(trifluoromethyl)biphenyl-2-yl]oxy}acetic acid (21)

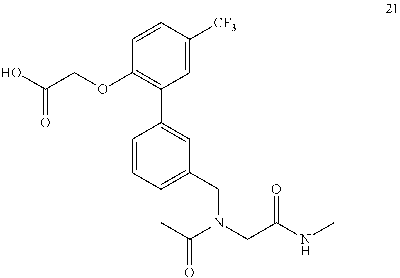

The procedure outlined in Example 17 was followed except the resultant secondary amine was acylated with acetic anhydride in the presence of triethylamine which upon deprotection yielded {[3′-({(acetyl)[2-(methylamino)-2-oxoethyl]amino}methyl)-5-(trifluoromethyl)biphenyl-2-yl]oxy}acetic acid (21) (18 mg, 45%) as a white solid. ¹HNMR (CD₃OD, 300 MHz): δ 7.64-7.50 (m, 3H), 7.50-7.35 (m, 2H), 7.28-7.21 (m, 1H), 7.14 (d, J=8.4 Hz, 1H), 4.78 (s, 2H), 4.71-4.63 (m, 2H), 4.00 (s, 2H), 2.72-2.68 (m, 3H), 2.25-2.12 (m, 3H). ¹⁹FNMR (CD₃OD, 300 MHz): −63.56. MS (ESI): MH⁺=439.1 HPLC (ZQ) t_{R}=5.50 min.

### Example 19

### {[3′-{[acetyl(phenyl)amino]methyl}-5-(trifluoromethyl)biphenyl-2-yl]oxy}acetic acid (22)

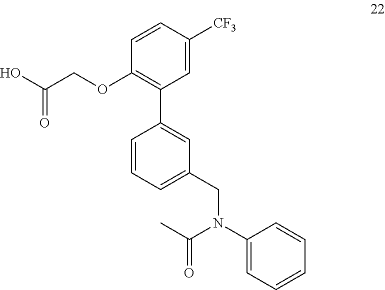

Reaction of aldehyde 16 (35 mg; 0.09 mmol) with aniline followed by acylation as in example 21 yielded {[3′-{[acetyl(phenyl)amino]methyl}-5-(trifluoromethyl)biphenyl-2-yl]oxy}acetic acid (22) (31 mg, 76%) as a white solid. ¹HNMR (CDCl₃, 300 MHz): δ 7.57-7.28 (m, 8H), 7.13-6.91 (m, 4H), 4.95 (s, 2H), 4.67 (s, 2H), 1.94 (s, 3H). ¹⁹FNMR (CDCl₃, 300 MHz): −62.02. MS (ESI): MH⁺=444.1. HPLC (ZQ) t_{R}=6.70 min.

### Example 20

### {[3′-{[methyl(phenylcarbonyl)amino]methyl}-5-(trifluoromethyl)biphenyl-2-yl]oxy}acetic acid (23)

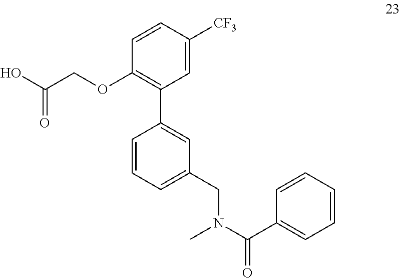

Following a similar procedure as in example 15, to a solution of amine 17 (0.05 g, 0.12 mmol, 1 eq) in anhydrous DCM (1 mL) was added K₂CO₃ (0.21 g, 1.54 mmol, 12 eq) and benzoyl chloride (0.09 mL, 0.77 mmol, 6 eq). The reaction was stirred at RT for 3 h, the solvent was evaporated, and the residue was purified by reverse phase preparative HPLC. This purified material was directly deprotected with 50% TFA/DCM for 2 h. The solvent was evaporated and the residue was purified by reverse phase preparative HPLC to give 41 mg (72%) of 23 as a white solid. ¹HNMR (CD₃OD, 300 MHz): δ 7.67-7.15 (m, 12H), 4.86-4.58 (m, 4H), 3.10-2.92 (m, 3H). ¹⁹FNMR (CD₃OD, 300 MHz): −63.46. MS (ESI): MH⁺=444.2 HPLC (ZQ) t_{R}=6.72 min.

### Example 21

### {[3′-{[methyl(phenoxycarbonyl)amino]methyl}-5-(trifluoromethyl)biphenyl-2-yl]oxy}acetic acid (24)

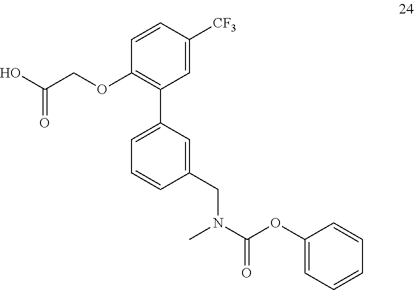

Following a similar procedure as in example 15, to a solution of amine 17 (0.05 g, 0.12 mmol, 1 eq) in anhydrous DCM (1 mL) was added K₂CO₃ (0.21 g, 1.54 mmol, 12 eq) and phenyl chloroformate (0.1 mL, 0.77 mmol, 6 eq). The reaction was stirred at RT for 3 h, the solvent was evaporated, and the residue was purified by reverse phase preparative HPLC. The purified material was directly deprotected with 50% TFA/DCM for 2 h. The solvent was evaporated and the residue was purified by reverse phase preparative HPLC to give 35 mg (59%) of 24 as a white solid. ¹HNMR (CDCl₃, 300 MHz): δ 7.64-7.40 (m, 4H), 7.35 (m, 5H), 7.23-7.04 (m, 2H) 6.93 (d, J=8.4 Hz, 1H), 4.75-4.58 (m, 4H), 3.12-2.97 (m, 3H). MS (ESI): MH⁺=460.1 HPLC (ZQ) t_{R}=7.25 min.

### Example 22

### {[3′-{[(butoxycarbonyl)(phenyl)amino]methyl}-5-(trifluoromethyl)biphenyl-2-yl]oxy}acetic acid (25)

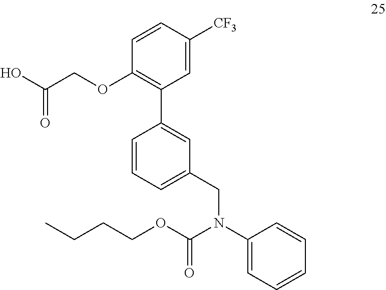

Following a similar procedure as in example 15, aldehyde 16 (0.05 g; 0.13 mmol) was reacted with aniline to generate a secondary amine intermediate which was directly reacted as follows. In anhydrous DCM (1 mL) was added K₂CO₃ (0.18 g, 1.31 mmol, 10 eq) and n-butyl chloroformate (0.08 mL, 0.66 mmol, 5 eq). The reaction was stirred at RT for 3 h, the solvent was evaporated, and the residue was purified by reverse phase preparative HPLC. This purified material was directly deprotected with 50% TFA/DCM for 2 h. The solvent was evaporated and the residue was purified by reverse phase preparative HPLC to give 59 mg (90%) of 25 as a white solid. ¹HNMR (CDCl₃, 300 MHz): δ 7.56-7.48 (m, 2H), 7.48-7.40 (m, 1H), 7.40-7.24 (m, 4H), 7.24-7.09 (m, 4H), 6.90 (d, J=8.4 Hz, 1H), 4.89 (s, 2H), 4.62 (s, 2H), 4.10 (t, J=6.6 Hz, 2H), 1.52 (m, 2H), 1.23 (m, 2H), 0.82 (t, J=7.2 Hz, 3H). ¹⁹FNMR (CDCl₃, 300 MHz): −62.00. MS (ESI): MH⁺=501.9 HPLC (LCQ) t_{R}=8.16 min.

### Example 23

### {[3′-{[(butoxycarbonyl)(1,3-thiazol-2-ylmethyl)amino]methyl}-5-(trifluoromethyl)biphenyl-2-yl]oxy}acetic acid (27)

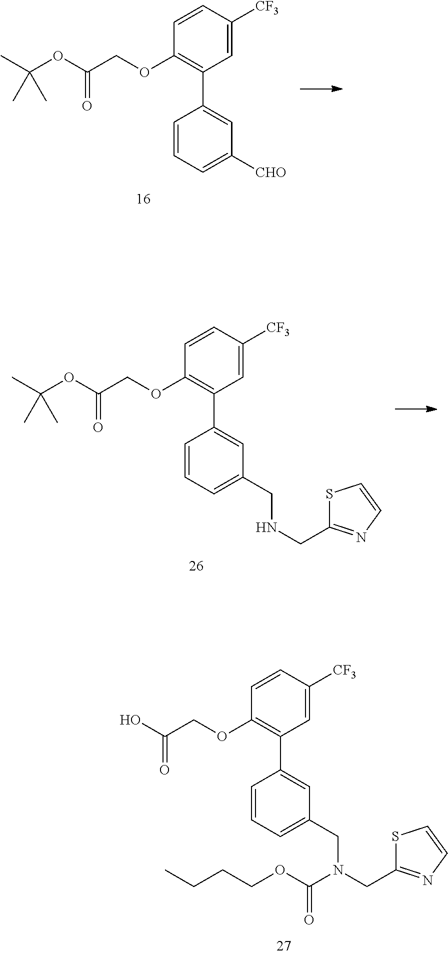

2-aminomethyl thiazole (0.07 g, 0.4 mmol, 3 eq) was treated with aldehyde 16 (0.05 g, 0.13 mmol, 1 eq) in anhydrous DCE and followed by NaBH(OAc)₃ (0.08 g, 0.4 mmol, 3 eq) as in example 18 to give secondary amine 26. Acylation with n-butyl chloroformate (0.08 mL, 0.66 mmol, 5 eq) and K₂CO₃ (0.18 g, 0.13 mmol, 10 eq) and subsequent ester-deprotection yields phenoxy acid 27 (42 mg, 61%) as a white solid. ¹HNMR (CDCl₃, 300 MHz): δ 7.88 (m, 1H), 7.78 (s, 1H), 7.63 (s, 1H), 7.58 (d, J=8.4 Hz, 1H), 7.50-7.28 (m, 4H), 6.97 (d, J=8.4 Hz, 1H), 4.81 (s, 2H), 4.63 (s, 2H), 4.56 (s, 2H), 4.26 (t, J=6.6 Hz, 2H), 1.70 (m, 2H), 1.41 (m, 2H), 0.94 (t, J=7.5 Hz, 3H). ¹⁹FNMR (CDCl₃, 300 MHz): −61.99. MS (ESI): MH⁺=523.2 HPLC (ZQ) t_{R}=7.53 min.

### Example 24

### {[3′-{[(anilinocarbonyl)(methyl)amino]methyl}-5-(trifluoromethyl)biphenyl-2-yl]oxy}acetic acid (28)

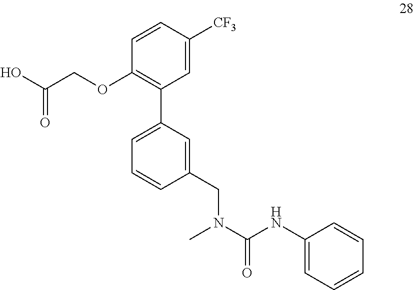

Following a similar procedure as in example 15, to a solution of amine 17 (0.13 mmol) in anhydrous DCM (1 mL) was added triethylamine (0.21 mL, 1.54 mmol, 12 eq) and phenyl isocyanate (0.08 mL, 0.77 mmol, 6 eq). The reaction was stirred at RT for 3 h, the solvent was evaporated, and the residue was purified by reverse phase preparative HPLC. The purified ester was treated directly with 50% TFA/DCM for 2 h. The solvent was evaporated and the residue was purified by reverse phase preparative HPLC to give 28 mg (47%) of 28 as a white solid. ¹HNMR (CDCl₃, 300 MHz): δ 7.65-7.40 (m, 5H), 7.23 (m, 5H), 7.02 (m, 1H) 6.87 (d, J=8.7 Hz, 1H), 4.64-4.50 (m, 4H), 3.00 (m, 3H). MS (ESI): MH⁺=459.2 HPLC (ZQ) t_{R}=6.68 min.

### Example 25

### {[3′-({methyl[(pyridine-2-ylamino)carbonyl]amino}methyl)-5-(trifluoromethyl)biphenyl-2-yl]oxy}acetic acid (29)

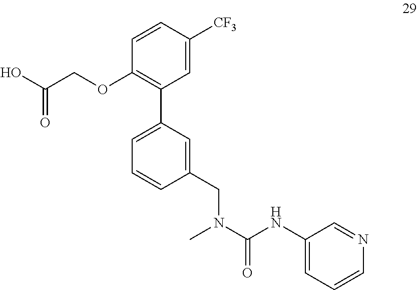

Following a similar procedure as in example 15, to a solution of amine 17 (0.13 mmol) in anhydrous DCM (1 mL) was added triethylamine (0.21 mL, 1.54 mmol, 12 eq) and 3-Pyridyl isocyanate (0.09 g, 0.77 mmol, 6 eq). The reaction was stirred at RT for 3 h, the solvent was evaporated, and the residue was purified by reverse phase preparative HPLC. The purified ester was treated directly with 50% TFA/DCM for 2 h. The solvent was evaporated and the residue was purified by reverse phase preparative HPLC to give 31 mg (52%) of 29 as a white solid. ¹HNMR (CDCl₃, 300 MHz): δ 9.46 (s, 1H), 8.97 (d, J=8.1 Hz, 1H), 8.16 (s, 1H), 8.11 (m, 2H), 7.76-7.70 (m, 2H), 7.64-7.32 (m, 3H), 7.24 (d, J=7.8 Hz, 1H), 6.98 (d, J=8.4 Hz, 1H), 4.76-4.62 (m, 4H), 3.54-3.04 (m, 3H). MS (ESI): MH⁺=460.1 HPLC (ZQ) t_{R}=5.12 min.

### Example 26

### {[3′-{[(butylaminocarbonyl)(methyl)amino]methyl}-5-(trifluoromethyl)biphenyl-2-yl]oxy}acetic acid (30)

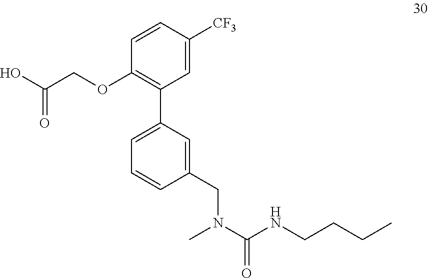

Following a similar procedure as in example 15, to a solution of amine 17 (0.04 g; 0.10 mmol) in anhydrous DCM (1 mL) was added n-butyl isocyanate (100 μL, 0.88 mmol, 8.8 eq). The reaction was stirred at RT for 16 h, the solvent was evaporated, and the residue was purified by silica gel chromatography (linear gradient of 0% EtOAC/hexanes to 100% EtOAC). The purified ester was treated directly with 50% TFA/DCM for 2 h. The solvent was evaporated and the residue was purified by reverse phase preparative HPLC to give 27 mg (60%) of 30. ¹HNMR (CDCl₃, 300 MHz): δ 7.55 (s, 2H), 7.50 (m, 1H), 7.39 (m, 2H), 7.18 (m, 1H), 6.89 (d, J=8.4 Hz, 2H), 4.59 (s, 2H), 4.51 (s, 2H), 3.19 (t, J=7.2 Hz, 2H), 2.90 (s, 3H), 1.39 (m, 2H), 1.22 (m, 2H), 0.83 (t, J=7.2 Hz, 3H). MS (ESI): MH⁺=439.2 HPLC (ZQ) t_{R}=6.54 min.

### Example 27

### {[3′-({methyl[(pyridine-2-yloxy)carbonyl]amino}methyl)-5-(trifluoromethyl)biphenyl-2-yl]oxy}acetic acid (31)

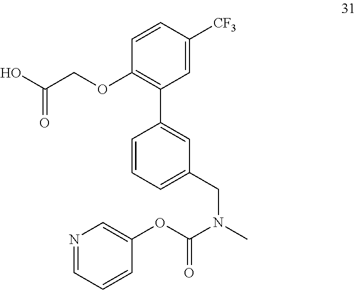

To a solution of triphosgene (0.03 g, 0.10 mmol, 1.1 eq) in anhydrous THF (0.8 ml), was slowly added a solution of 3-hydroxy pyridine (0.02 g, 0.21 mmol, 2.3 eq) and DIEA (0.06 mL, 0.35 mmol, 3.8 eq). The mixture was pre-stirred at RT for 20 min. prior to the addition of amine 17 (0.03 g, 0.09 mmol, 1 eq) in anhydrous THF (0.5 mL). After stirring for 16 h, the solvent was evaporated and the residue was purified by reverse phase preparative HPLC to give 30 mg (64%) of ester. The ester was treated with 50% TFA/DCM for 2 h, the solvent was evaporated, and the residue was purified by reverse phase preparative HPLC to give 17 mg (63%) of 31 as a white solid. ¹HNMR (CDCl₃, 300 MHz): δ 9.48-9.24 (m, 1H), 8.70-8.55 (m, 1H), 8.28-7.92 (m, 1H), 7.92-7.77 (m, 2H), 7.60-7.52 (m, 2H), 7.50-7.36 (m, 2H), 7.28-7.20 (m, 1H), 6.98 (dd, J=8.4 Hz, 1H), 4.8-4.65 (m, 4H), 3.09-3.01 (m, 3H). ¹⁹FNMR (CDCl₃, 300 MHz): −61.95. MS (ESI): MH⁺=461.1 HPLC (ZQ) t_{R}=5.78 min.

### Example 28

### {[3′-{[methyl(pyridine-3-ylcarbonyl)amino]methyl}-5-(trifluoromethyl)biphenyl-2-yl]oxy}acetic acid (32)

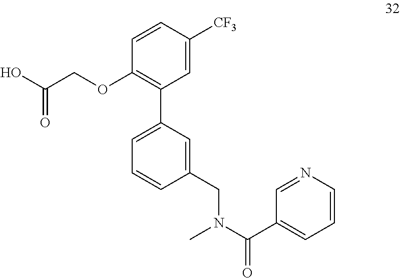

To a solution of amine 17 (0.04 g, 0.09 mmol, 1 eq) in DCM (1 mL) was added nicotinic acid (0.02 g, 0.5 mmol, 2 eq), and EDC (0.03 g, 0.25 mmol, 2 eq). After stirring at RT for 48 h, the solvent was evaporated and the residue was purified by reverse phase preparative HPLC to give the protected ester. This material was directly treated with 30% TFA/DCM for 2 h. The solvent was evaporated and the residue was purified by reverse phase preparative HPLC to give 25.8 mg (67%) of 32 as a white solid. ¹HNMR (CDCl₃, 300 MHz): δ 9.64 (s, 1H), 8.96 (d, J=5.4 Hz, 1H), 8.58 (d, J=7.8 Hz, 1H), 7.99 (dd, J=6.6, 5.4 Hz, 1H), 7.78 (s, 1H), 7.64-53 (m, 2H), 7.47 (dd, J=7.5 Hz, 1H), 7.41-7.28 (m, 2H), 7.00 (d, J=8.7 Hz, 1H), 4.90-4.54 (m, 4H), 3.04 (m, 3H). ¹⁹FNMR (CDCl₃, 300 MHz): −61.99. MS (ESI): MH⁺=445.1 HPLC (ZQ) t_{R}=5.52 min.

### Example 29

### {[3′-{[(benzensulfonyl)(methyl)amino]methyl}-5-(trifluoromethyl)biphenyl-2-yl]oxy}acetic acid (33)

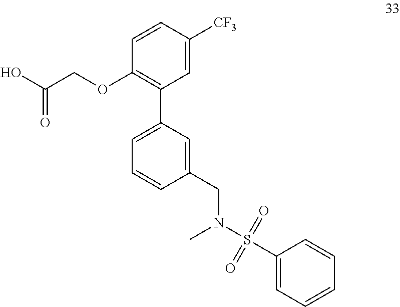

To a solution of amine 17 (0.033 g; 0.08 mmol) in anhydrous DCM (1 mL) was added DIEA (100 μL; 0.57 mmol; 7 eq) and benzenesulfonyl chloride (30 μL, 0.12 mmol, 1.5 eq). The reaction was stirred at RT for 16 h, the solvent was evaporated, and the residue was purified by silica gel chromatography (linear gradient of 0% EtOAC/hexanes to 100% EtOAC). The purified ester was treated directly with 30% TFA/DCM for 2 h. The solvent was evaporated and the residue was purified by reverse phase preparative HPLC to give 22 mg (57%) of 33. ¹HNMR (CDCl₃, 300 MHz): δ 7.82 (m, 2H), 7.56 (m, 6H), 7.41 (m, 2H), 7.28 (d, J=7.8 Hz, 1H), 6.93 (d, J=9.3 Hz, 1H), 4.69 (s, 2H), 4.19 (s, 2H), 2.63 (s, 3H), MS (ESI): MH⁺=480.3 HPLC (ZQ) t_{R}=7.22 min.

### Example 30

### {[3′-{[(pyridine-3-ylsulfonyl)(methyl)amino]methyl}-5-(trifluoromethyl)biphenyl-2-yl]oxy}acetic acid (34)

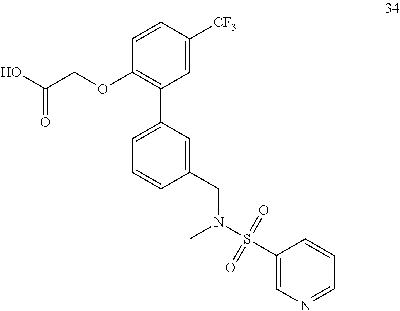

To a solution of amine 17 (0.033 g; 0.08 mmol) in anhydrous DCM (1 mL) was added DIEA (100 μL; 0.57 mmol; 7 eq) and pyridine-3-sulfonyl chloride hydrochloride (30 μL, 0.12 mmol, 1.5 eq). The reaction was stirred at RT for 16 h, the solvent was evaporated, and the residue was purified by silica gel chromatography (linear gradient of 0% EtOAC/hexanes to 100% EtOAC). The purified ester was treated directly with 30% TFA/DCM for 2 h. The solvent was evaporated and the residue was purified by reverse phase preparative HPLC to give 21 mg (55%) of 34. ¹HNMR (CDCl₃, 300 MHz): δ 9.05 (bs, 1H), 8.82 (bs, 1H), 8.19 (d, J=8.1 Hz, 1H), 7.56 (m, 4H), 7.40 (m, 2H), 7.28 (d, J=7.2 Hz, 1H), 6.96 (d, J=9.3 Hz, 1H), 4.70 (s, 2H), 4.28 (s, 2H), 2.73 (s, 3H) MS (ESI): MH⁺=481.0 HPLC (ZQ) t_{R}=8.18 min.

### Example 31

### {[4′-({(butoxy carbonyl)[2-(butylamino)-2-oxoethyl]amino}ethyl)biphenyl-2-yl]oxy}acetic acid (36)

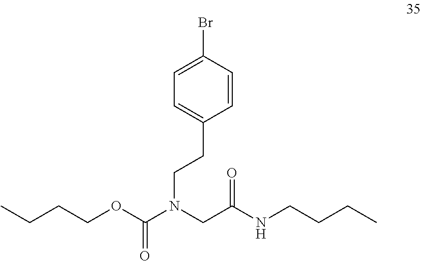 [0210] b) Following the method of example 1 except replacing 3-iodobenzylamine with 4-bromophenethyl amine generates intermediate butyl 4-bromophenethyl(2-(butylamino)-2-oxoethyl)carbamate (35) (0.4 g, 28%). ¹HNMR (CDCl₃, 300 MHz): δ 7.41 (d, J=8.4 Hz, 2H), 7.05 (m, 2H), 4.09 (t, J=6.6 Hz, 2H), 3.79 (s, 2H), 3.52 (t, J=7.2 Hz, 2H), 3.21 (m, 2H), 2.82 (t, J=7.2 Hz, 2H), 1.65-1.52 (m, 2H), 1.50-1.23 (m, 6H), 0.94 (t, J=7.2 Hz, 3H), 0.91 (t, J=7.5 Hz, 3H).

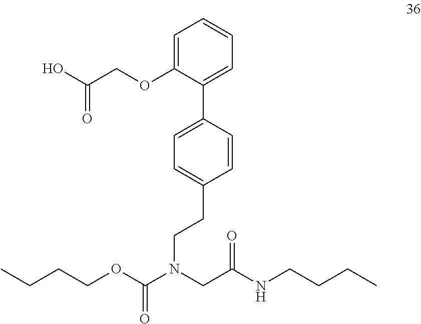

c) Upon further treatment of 35 as in example 6, {[4′-({(butoxy carbonyl)[2-(butylamino)-2-oxoethyl]amino}ethyl)biphenyl-2-yl]oxy}acetic acid (36) was obtained (0.016 g; 25%) as a white solid. ¹HNMR (CDCl₃, 300 MHz): δ 7.44 (d, J=8.1 Hz, 2H), 7.34-7.27 (m, 2H), 7.19 (d, J=8.4 Hz, 2H), 7.09 (dd, J=8.7, 6.9 Hz, 1H), 6.94 (d, J=8.7 Hz, 1H), 4.48 (s, 2H), 4.13 (m, 2H), 3.56 (m, 4H), 3.21 (m, 2H), 2.91 (bs, 2H), 1.61 (bs, 2H), 1.48-1.20 (m, 6H), 1.0-0.82 (m, 6H). MS (ESI): MH⁺=485.3. HPLC (ZQ) t_{R}=6.78 min.

Analytical HPLC Analysis:

Method A: Waters Millenium 2695/996PDA separations system employing a Phenomenex Columbus 5μ C18 110A column 100×2.0 mm analytical column. The aqueous acetonitrile based solvent gradient involves;

0-0.5 min—Isocratic 10% of (0.1% TFA/acetonitrile); 0.5 min-5.5 min—Linear gradient of 10-80% of (0.1% TFA/acetonitrile): 5.5 min-7.5 min—Isocratic 80% of (0.1% TFA/acetonitrile); 7.5 min-8 min—Linear gradient of 80-10% of (0.1% TFA/acetonitrile); 8 min-10 min—Isocratic 10% of (0.1% TFA/acetonitrile). Flow rate=0.3 mL/min.

Method B: Waters Millenium 2695/996PDA separations system employing a Phenomenex Columbus 5μ C18 110A column 100×2.0 mm analytical column. The aqueous acetonitrile based solvent gradient involves;

0-0.5 min—Isocratic 10% of (0.05% TFA/acetonitrile); 0.5 min-5.5 min—Linear gradient of 10-90% of (0.05% TFA/acetonitrile): 5.5 min-7.5 min—Isocratic 90% of (0.05% TFA/acetonitrile); 7.5 min-8 min—Linear gradient of 90-10% of (0.05% TFA/acetonitrile); 8 min-10 min—Isocratic 10% of (0.05% TFA/acetonitrile). Flow rate=0.4 mL/min.

Semi-Preparative HPLC Purification

Semi-preparative HPLC purification was conducted on a Waters 600 Semi-preparative liquid chromatograph equipped with a 996 diode array detector employing a Sunfire™ Prep C18 OBD 5 μm 19×100 mm column. The aqueous acetonitrile based solvent gradient involves;

0 min-9 min—Linear gradient of 10-90% of (0.05% TFA/acetonitrile): 9 min-10 min—Isocratic 90% of (0.05% TFA/acetonitrile); 10 min-10.5 min—Linear gradient of 90-10% of (0.05% TFA/acetonitrile).

Mass Spectroscopy

Mass Spectroscopy was conducted using an Applied Biosciences PE Sciex API150ex. Liquid Chromatography Mass Spectroscopy was conducted using a Waters Millenium 2695/996PDA linked Thermo-electron LCQ classic or a Waters Micromass ZQ utilizing a Waters 1525 HPLC pump.

NMR Spectroscopy

¹H NMR spectroscopy was conducted using a Varian 300 MHz Gemini 2000 FTNMR.

Chemical Structure Naming

Names for each aryl fragment in the biaryl structure were generated using “Convert Structure to Name” function in ChemDraw version 8.8.0 (ChembridgeSoft Corporation, MA, USA). The final biaryl compounds were named based on IUPAC convention.

[³⁵S]GTPγS Binding Assay

SPA [³⁵S]GTPγS binding was performed on membranes from CHO-K1 cells stably expressing human CRTH2. To a white, clear-bottomed 384-well plate, 4 μg membrane protein was incubated in the binding buffer (20 mM HEPES, pH 7.4, 10 mM MgCl₂, 300 mM NaCl, 0.2% BSA) with 60 nM DHK-PGD₂, 500 pM [³⁵S]GTPγS, 10 μM GDP, and 100 μg wheat germ agglutinin-coupled SPA beads (GE Healthcare) with and without increasing concentrations of compounds. The final assay volume was 40 μL and contained 1% DMSO. Samples were incubated for 2 h at ambient temperature. Plates were centrifuged 5 min at 1500 rpm. Plates were read after an additional 4 h using the Trilux 1450 Microbeta (PerkinElmer) with a 30 sec/well reading time.

It has been established that compounds that are active in the GTPγS assay have the potential to trigger an in vivo response (Mathiesen et al., 2006, Mol. Pharmacology 69: 1441-1453; Uller et al., 2007, Respiratory Research 8:16).

Representative species are shown below in Table 1. Compounds exhibited IC₅₀ values for CRTH2<1 μM. Potency of compound is further divided into two groups: ++, IC₅₀<100 nM; +, IC₅₀≧100 nM to <1 μM

TABLE 1

| | | | | CRTH2 |
|---|---|---|---|---|
| | | HPLC retention | | GTPγS |
| Compound | Structure | time/(method) | [M + H]+ | assay IC₅₀ |
| 6 | 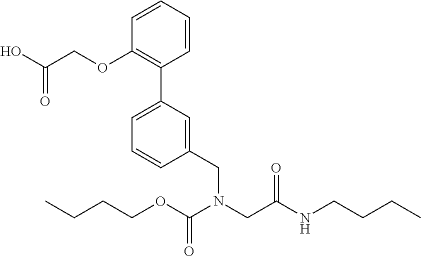 | 6.73 min (Method A) | 471.1 | ++ |
| | {[3′-({(butoxy carbonyl)[2- (butylamino)-2- oxoethyl]amino}methyl)biphenyl-2- yl]oxy}acetic acid | | | |
| 7 | 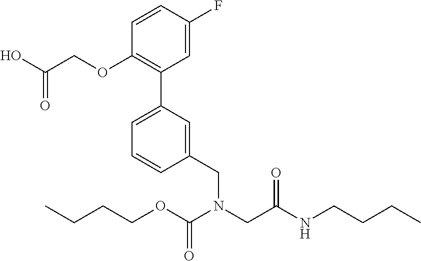 | 7.15 min (Method B) | 489.0 | ++ |
| | {[3′-({(butoxy carbonyl)[2- (butylamino)-2- oxoethyl]amino}methyl)-5- fluorobiphenyl-2-yl]oxy}acetic acid | | | |
| 8 | 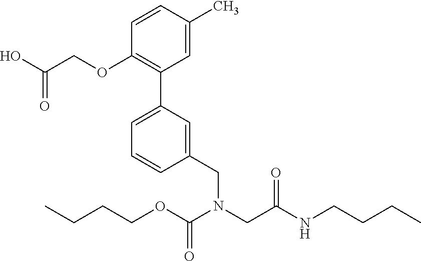 | 6.98 min (Method A) | 484.0 | ++ |
| | {[3′-({(butoxy carbonyl)[2- (butylamino)-2- oxoethyl]amino}methyl)-5- methylbiphenyl-2-yl]oxy}acetic acid | | | |
| 9 | 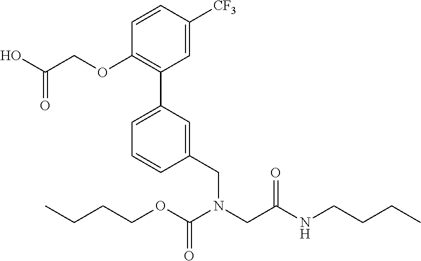 | 7.25 min (Method A) | 538.0 | ++ |
| | {[3′-({(butoxy carbonyl)[2- (butylamino)-2- oxoethyl]amino}methyl)-5- (trifluoromethyl)biphenyl-2- yl]oxy}acetic acid | | | |
| 11 | 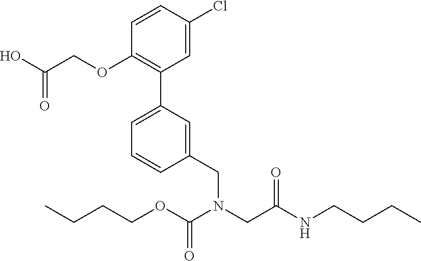 | 7.13 min (Method A) | 504.0 | ++ |
| | {[3′-({(butoxy carbonyl)[2- (butylamino)-2- oxoethyl]amino}methyl)-5- chlorobiphenyl-2-yl]oxy}acetic acid | | | |
| 13 | 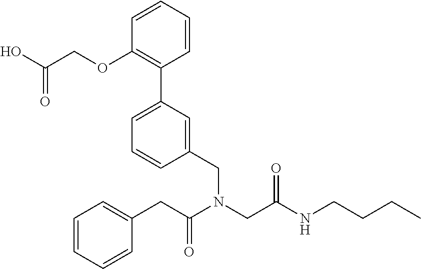 | 6.42 min (Method A) | 488.0 | ++ |
| | {[3′-{[[2-(butylamino)-2- oxoethyl](phenylacetyl)amino]methyl}- biphenyl-2-yl]oxy}acetic acid | | | |
| 18 | 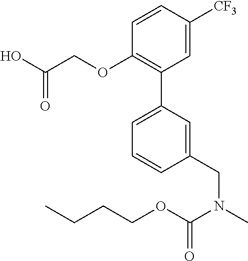 | 7.50 min (Method A) | 439.0 | ++ |
| | {[3′- {[(butoxycarbonyl)(methyl)amino] methyl}-5-(trifluoromethyl)biphenyl- 2-yl]oxy}acetic acid | | | |
| 19 | 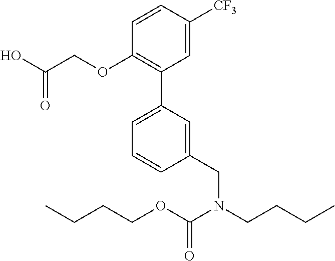 | 9.50 min (Method A) | 482.1 | ++ |
| | {[3′- {[(butoxycarbonyl)(butyl)amino] methyl}-5-(trifluoromethyl)biphenyl- 2-yl]oxy}acetic acid | | | |
| 20 | 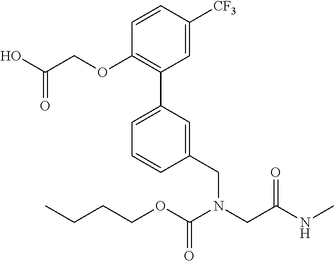 | 6.55 min (Method A) | 497.1 | ++ |
| | {[3′-({(butoxycarbonyl) [2- (methylamino)-2- oxoethyl]amino}methyl)-5- (trifluoromethyl)biphenyl-2- yl]oxy}acetic acid | | | |
| 21 | 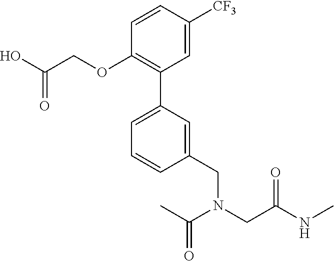 | 5.50 min (Method A) | 439.1 | + |
| | {[3′-({(acetyl)[2-(methylamino)-2- oxoethyl]amino}methyl)-5- (trifluoromethyl)biphenyl-2- yl]oxy}acetic acid | | | |
| 22 | 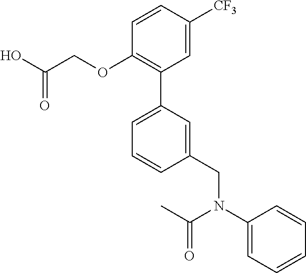 | 6.70 min (Method A) | 444.1 | + |
| | {[3′- {[acetyl(phenyl)amino]methyl}-5- (trifluoromethyl)biphenyl-2- yl]oxy}acetic acid | | | |
| 23 | 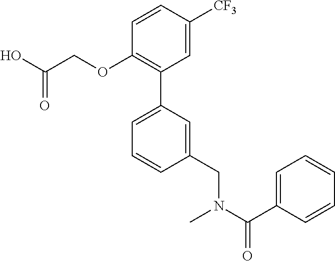 | 6.72 min (Method A) | 444.2 | + |
| | {[3′- {[methyl(phenylcarbonyl)amino] methyl}-5-(trifluoromethyl)biphenyl-2- yl]oxy}acetic acid | | | |
| 24 | 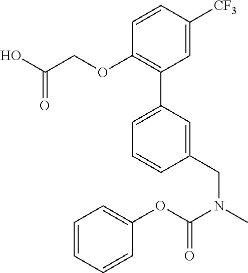 | 7.25 min (Method A) | 460.1 | + |
| | {[3′- {[methyl(phenoxycarbonyl)amino] methyl}-5-(trifluoromethyl)biphenyl- 2-yl]oxy}acetic acid | | | |
| 25 | 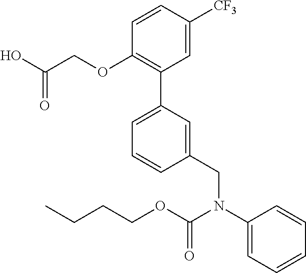 | 8.16 min (Method B) | 501.9 | + |
| | {[3′- {[(butoxycarbonyl)(phenyl)amino] methyl}-5-(trifluoromethyl)biphenyl- 2-yl]oxy}acetic acid | | | |
| 27 | 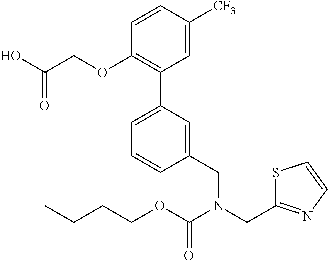 | 7.53 min (Method A) | 523.2 | ++ |
| | {[3′-{[(butoxycarbonyl)(1,3-thiazol- 2-ylmethyl)amino]methyl}-5- (trifluoromethyl)biphenyl-2- yl]oxy}acetic acid | | | |
| 28 | 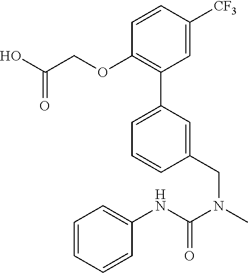 | 6.68 min (Method A) | 459.2 | + |
| | {[3′- {[(anilinocarbonyl)(methyl)amino] methyl}-5-(trifluoromethyl)biphenyl- 2-yl]oxy}acetic acid | | | |
| 29 | 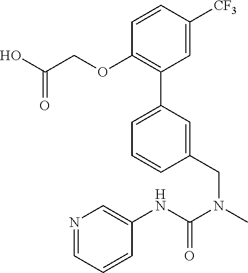 | 5.12 min (Method A) | 460.1 | + |
| | {[3′-({methyl[(pyridine-2- ylamino)carbonyl]amino}methyl)-5- (trifluoromethyl)biphenyl-2- yl]oxy}acetic acid | | | |
| 30 | 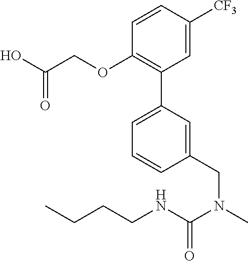 | 6.54 min (Method A) | 439.2 | ++ |
| | {[3′- {[(butylaminocarbonyl)(methyl) amino]methyl}-5- (trifluoromethyl)biphenyl-2- yl]oxy}acetic acid | | | |
| 31 | 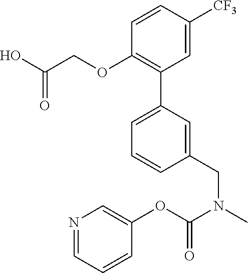 | 5.78 min (Method A) | 461.1 | + |
| | {[3′-({methyl[(pyridine-2- yloxy)carbonyl]amino}methyl)-5- (trifluoromethyl)biphenyl-2- yl]oxy}acetic acid | | | |
| 32 | 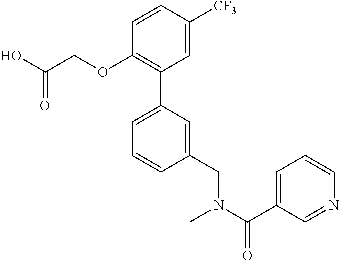 | 5.52 min (Method A) | 445.1 | ++ |
| | {[3′-{[methyl(pyridine-3- ylcarbonyl)amino]methyl}-5- (trifluoromethyl)biphenyl-2- yl]oxy}acetic acid | | | |
| 33 | 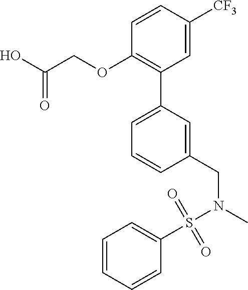 | 7.22 min (Method A) | 480.3 | + |
| | {[3′- {[(benzenesulfonyl)(methyl)amino] methyl}-5-(trifluoromethyl)biphenyl- 2-yl]oxy}acetic acid | | | |
| 34 | 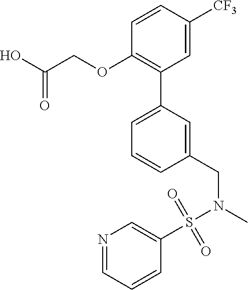 | 8.18 min (Method A) | 481.0 | + |
| | {[3′-{[(pyridine-3- ylsulfonyl)(methyl)amino]methyl}- 5-(trifluoromethyl)biphenyl-2- yl]oxy}acetic acid | | | |
| 36 | 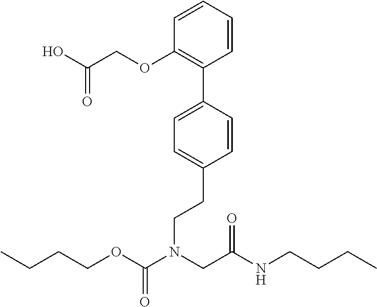 | 6.78 min (Method A) | 485.3 | ++ |
| | {[4′-({(butoxy carbonyl)[2- (butylamino)-2- oxoethyl]amino}ethyl)biphenyl-2- yl]oxy}acetic acid | | | |

1. A compound of formula I 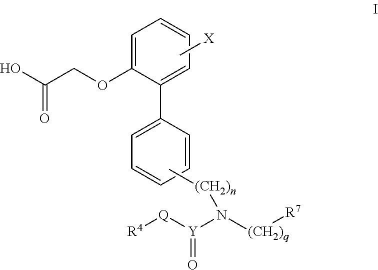
or a salt thereof
wherein
X is selected from the group consisting of hydrogen, halogen, cyano, (C₁-C₄)alkyl, —O(C₁-C₄)alkyl and —S(O)ₘ(C₁-C₄)alkyl, each (C₁-C₄)alkyl optionally substituted with one chlorine, iodine or bromine atom or one or more fluorine atoms;
m is zero, one or two;
n is one or two;
Y is carbon or SO;
Q is oxygen, NH or (CH₂)_{P}, with the proviso that when Y is SO, Q cannot be oxygen;
p is zero or 1-4;
R⁴ is selected from the group consisting of:
(a) aryl and heterocyclyl, each optionally substituted with one to three substituents chosen from (C₁-C₄)alkyl, (C₁-C₄)alkoxy, (C₁-C₄)haloalkyl, halogen, cyano and (C₁-C₄)haloalkoxy;
(b) (C₁-C₈)alkyl, optionally substituted with one to three substituents chosen from the group consisting of (C₁-C₈)alkyl, (C₁-C₈)haloalkyl, chlorine, iodine, bromine, cyano and (C₁-C₈)haloalkoxy or optionally substituted with one or more fluorine atoms; and
(c) (C₁-C₈)heteroalkyl;
q is zero or 1-4; and
R⁷ is chosen from:
(a) aryl and heterocyclyl, each optionally substituted with one to three substituents from the group consisting of (C₁-C₄)alkyl, (C₁-C₄)alkoxy, (C₁-C₄)haloalkyl, halogen, cyano and (C₁-C₄)haloalkoxy;
(b) (C₁-C₈)alkyl, optionally substituted with one to three substituents from the group consisting of (C₁-C₈)alkyl, (C₁-C₈)haloalkyl, chlorine, iodine, bromine, cyano and (C₁-C₈)haloalkoxy or optionally substituted with one or more fluorine atoms;
(c) (C₁-C₈)heteroalkyl; and
(d) CON(H) (C₁-C₈)alkyl; with the proviso that when Q is (CH₂)_{P}, R⁴ and R⁷ cannot both be alkyl.

2. A compound or salt according to claim 1 wherein n is one.

3. A compound or salt according to claim 1 wherein the (CH₂)ₙ substituent is in the meta position.

4. A compound or salt according to claim 1 wherein Y is equal to carbon.

5. A compound or salt according to claim 4 wherein Q is equal to oxygen and R⁴ is alkyl, heteroaryl or aryl.

6. A compound or salt according to claim 5 wherein R⁴ is butyl.

7. A compound or salt according to claim 1 wherein q is one.

8. A compound or salt according to claim 7 wherein R⁷ is CON(H)alkyl.

9. A compound or salt according to claim 8 wherein R⁷ is CON(H)butyl.

10. A compound or salt according to claim 1 wherein q is zero and R⁷ is methyl.

11. A compound or salt according to claim 3 wherein
X is selected from the group consisting of hydrogen, halogen, CF₃ and alkyl; n is one;
Y is carbon;
Q is oxygen;
R⁴ is a (C₁-C₄)alkyl;
q is one; and
R⁷ is chosen from:
(a) aryl and heterocyclyl, each optionally substituted with one to three substituents from the group consisting of (C₁-C₄)alkyl, (C₁-C₄)alkoxy, haloalkyl, halogen, cyano and (C₁-C₄)haloalkoxy;
(b) (C₁-C₈)alkyl, optionally substituted with one to three substituents from the group consisting of (C₁-C₈)alkyl, (C₁-C₈)haloalkyl, chlorine, iodine, bromine, cyano and (C₁-C₈)haloalkoxy or optionally substituted with one or more fluorine atoms;
(c) heteroalkyl; and
(d) CON(H)(C₁-C₈)alkyl.

12. A compound or salt according to claim 11 wherein R⁷ is CON(H)alkyl.

13. A compound or salt according to claim 3 of formula II 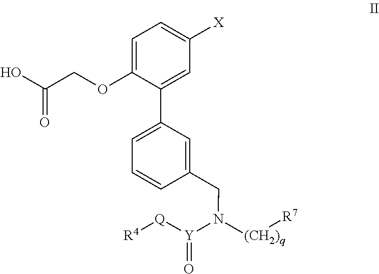
wherein X is selected from the group consisting of hydrogen, halogen, CF₃ and alkyl.

14. A compound or salt according to claim 1 wherein Y is equal to SO.

15. A compound or salt according to claim 14 wherein Q is (CH₂)_{P}, p is zero, and R⁴ is an aryl or a heteroaryl.

16. A compound or salt according to claim 1 wherein the (CH₂)ₙ substituent is in the para position.

17. A compound or salt according to claim 1 wherein X is hydrogen, alkyl, CF₃, chlorine or fluorine.

18. A compound or salt according to claim 17 wherein X is methyl.

19. A compound or salt according to claim 1 wherein X is CF₃.

20. A compound or salt according to claim 19 wherein R⁷ is aryl or heteroaryl.

21. A compound or salt according to claim 1 wherein R⁴ is selected from phenyl and pyridine.

22. A compound or salt according to claim 1 wherein R⁷ is selected from phenyl and thiazole.

23. A compound or salt thereof according to claim 1 selected from
{[3′-({(butoxycarbonyl) [2-(butylamino)-2-oxoethyl]amino}methyl)biphenyl-2-yl]oxy}acetic acid,
{[3′-({(butoxycarbonyl)[2-(butylamino)-2-oxoethyl]amino}methyl)-5-fluorobiphenyl-2-yl]oxy}acetic acid,
{[3′-({(butoxycarbonyl)[2-(butylamino)-2-oxoethyl]amino}methyl)-5-methylbiphenyl-2-yl]oxy}acetic acid,
{[3′-({(butoxycarbonyl) [2-(butylamino)-2-oxoethyl]amino}methyl)-5-(trifluoromethyl)biphenyl-2-yl]oxy}acetic acid,
{[3′-({(butoxycarbonyl) [2-(butylamino)-2-oxoethyl]amino}methyl)-5-chlorobiphenyl-2-yl]oxy}acetic acid,
{[3′-{[[2-(butylamino)-2-oxoethyl] (phenylacetyl)amino]methyl}-biphenyl-2-yl]oxy}acetic acid,
{[3′-{[(butoxycarbonyl)(methyl)amino]methyl}-5-(trifluoromethyl)biphenyl-2-yl]oxy}acetic acid,
{[3′-{[(butoxycarbonyl)(butyl)amino]methyl}-5-(trifluoromethyl)biphenyl-2-yl]oxy}acetic acid,
{[3′-({(butoxycarbonyl)[2-(methylamino)-2-oxoethyl]amino}methyl)-5-(trifluoromethyl)biphenyl-2-yl]oxy}acetic acid,
{[3′4 {(acetyl) [2-(methylamino)-2-oxoethyl]amino}methyl)-5-(trifluoromethyl)biphenyl-2-yl]oxy}acetic acid,
{[3′-{[acetyl(phenyl)amino]methyl}-5-(trifluoromethyl)biphenyl-2-yl]oxy}acetic acid,
{[3′-{[methyl(phenylcarbonyl)amino]methyl}-5-(trifluoromethyl)biphenyl-2-yl]oxy}acetic acid,
{[3′-{[methyl(phenoxycarbonyl)amino]methyl}-5-(trifluoromethyl)biphenyl-2-yl]oxy}acetic acid,
{[3′-{[(butoxycarbonyl)(phenyl)amino]methyl}-5-(trifluoromethyl)biphenyl-2-yl]oxy}acetic acid,
{[3′-{[(butoxycarbonyl)(1,3-thiazol-2-ylmethyl)amino]methyl}-5-(trifluoromethyl)biphenyl-2-yl]oxy}acetic acid,
{[3′-{[(anilinocarbonyl)(methyl)amino]methyl}-5-(trifluoromethyl)biphenyl-2-yl]oxy}acetic acid,
{[3′-({methyl[(pyridine-2-ylamino)carbonyl]amino}methyl)-5-(trifluoromethyl)biphenyl-2-yl]oxy}acetic acid,
{[3′-{[(butylaminocarbonyl)(methyl)amino]methyl}-5-(trifluoromethyl)biphenyl-2-yl]oxy}acetic acid,
{[3′4 {methyl[(pyridine-2-yloxy)carbonyl]amino}methyl)-5-(trifluoromethyl)biphenyl-2-yl]oxy}acetic acid,
{[3′-{[methyl(pyridine-3-ylcarbonyl)amino]methyl}-5-(trifluoromethyl)biphenyl-2-yl]oxy}acetic acid,
{[3′-{[(benzensulfonyl)(methyl)amino]methyl}-5-(trifluoromethyl)biphenyl-2-yl]oxy}acetic acid,
{[3′-{[(pyridine-3-ylsulfonyl)(methyl)amino]methyl}-5-(trifluoromethyl)biphenyl-2-yl]oxy}acetic acid, and
{[4′-({(butoxy carbonyl) [2-(butylamino)-2-oxoethyl]amino}ethyl)biphenyl-2-yl]oxy}acetic acid.

24. A pharmaceutical composition comprising a pharmaceutically acceptable carrier and a therapeutically effective amount of at least one compound or salt according to claim 1.

25. A method of treating, preventing or ameliorating a disorder responsive to inhibition of chemoattractant receptor-homogolous molecule expressed on T helper 2 cells, which comprises administering to a subject in need of such treatment a therapeutically effective amount of a compound or salt according to claim 1.

26. A method according to claim 25 wherein said disorder is an inflammatory disease.

27. A method according to claim 25 wherein said disorder is a respiratory disease.

28. A method according to claim 25 wherein said disorder is selected from asthma, rhinitis, chronic obstructive pulmonary disease, bronchitis, nasal polyposis, nasal congestion, farmer's lung, fibroid lung and cough.

29. A method according to claim 25 wherein said disorder is a skin disorder.

30. A method according to claim 25 wherein said disorder is dermatitis, cutaneous eosinophilias, Lichen planus, urticaria, psoriasis, pruritus, angiodermas, corneal ulcers, chronic skin ulcers, conjunctivitis, vasculitides, uveitis or erythemas.

31. A method according to claim 25 wherein said disorder is selected from osteoarthritis, rheumatoid arthritis, pain and inflammatory bowel disease.

32. A method according to claim 25 wherein said subject is a human.

33. A salt of a compound according to claim 1 wherein said salt is a pharmaceutically acceptable salt.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)
